(19) United States
(12) Patent Application Publication
(10) Pub. No.: US 2025/0084058 A1
(43) Pub. Date: Mar. 13, 2025

(54) PHENYLPYRAZOLE COMPOUND AND APPLICATION THEREOF

(71) Applicants: METISA BIOTECHNOLOGY CO., LTD., Nanning (CN); SHENYANG UNIVERSITY OF CHEMICAL TECHNOLOGY, Shenyang (CN)

(72) Inventors: Lixin ZHANG, Shenyang (CN); Jing ZHANG, Shenyang (CN); Dongdong LIU, Shenyang (CN); Zhuo KANG, Shenyang (CN)

(73) Assignees: METISA BIOTECHNOLOGY CO., LTD., Nanning (CN); SHENYANG UNIVERSITY OF CHEMICAL TECHNOLOGY, Shenyang (CN)

(21) Appl. No.: 18/941,908

(22) Filed: Nov. 8, 2024

Related U.S. Application Data
(63) Continuation of application No. PCT/CN2023/092730, filed on May 8, 2023.

(30) Foreign Application Priority Data
May 10, 2022 (CN) 202210502001.0

(51) Int. Cl. C07D 401/04; A01N 43/56; A01P 7/02; A01P 7/04; A61K 31/4439; A61K 45/06; A61P 33/14

(52) U.S. Cl. CPC C07D 401/04; A01N 43/56; A01P 7/02; A01P 7/04; A61K 31/4439; A61K 45/06; A61P 33/14

(57) ABSTRACT

The present disclosure discloses a phenylpyrazole compound and an application thereof. The structure of the compound is as shown in general formula (I),

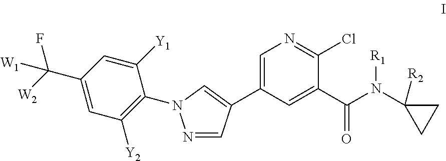

and the definitions of the various substituents in the formula are given in the description. The description also discloses a use of the compound as an insecticide and acaricide, and as an animal parasite control agent.

### CROSS-REFERENCE TO RELATED APPLICATIONS

The present disclosure is a Continuation Application of International Application No. PCT/CN2023/092730, filed on May 8, 2023, which claims priority to the Chinese Patent Application No. 202210502001.0 filed on May 10, 2022, the entire contents of which are incorporated herein by reference.

### TECHNICAL FIELD

The present disclosure relates to a phenylpyrazole compound, and in particular to a novel phenylpyrazole compound and an application thereof.

### BACKGROUND

Patents CN105873906A and CN106103414A relate to some phenylpyrazole compounds with insecticidal and acaricidal activity. However, since pests and acarids have resistance after insecticides and acaricides are used for a period of time, it is necessary to continuously develop novel and improved compounds with insecticidal and acaricidal activity. Meanwhile, with the increasing need for agricultural and livestock products and the increasing attention to environmental protection, there has always been a need to use novel insecticides and acaricides with high efficiency, broad spectrum and environmental friendliness.

### SUMMARY

The purpose of the present disclosure is to provide a phenylpyrazole compound with better insecticidal and acaricidal activity and an application thereof. The phenylpyrazole compound can be used for preparing a drug for prevention and control of pests and acarids in agriculture and other fields as well as for preparing a drug for control of animal parasites in the field of veterinary drugs.

In order to achieve the purpose of the present disclosure, the present disclosure provides the following technical solutions.

A phenylpyrazole compound is as shown in general formula I, or is a stereoisomer of the compound as shown in the general formula I; a salt of the compound as shown in the general formula I; or a salt of the stereoisomer of the compound as shown in the general formula I:

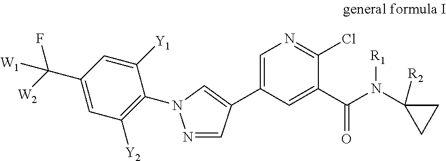

where in the general formula I:
W₁ is selected from fluorine, trifluoromethyl, or pentafluoroethyl;
W₂ is selected from fluorine or trifluoromethyl;
Y₁ is selected from halogen;
Y₂ is selected from halogen, C₁-C₃ haloalkyl, or C₁-C₃ haloalkoxy;
R₁ is selected from allyl, propargyl, cyclopropylmethyl, CNCH₂—, CNCH₂CH₂—, CNCH₂CH₂CH₂—, CNCH₂CH₂CH₂CH₂—, or

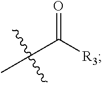

R₂ is selected from hydrogen or cyanide; and
R₃ is selected from C₁-C₄ alkyl, C₃-C₆ cycloalkyl, or C₁-C₄ alkoxy C₁-C₃ alkyl.

In a possible implementation, in the general formula I,
W₁ is selected from fluorine, trifluoromethyl, or pentafluoroethyl;
W₂ is selected from fluorine or trifluoromethyl;
Y₁ is selected from chlorine or bromine;
Y₂ is selected from chlorine, bromine, C₁-C₃ haloalkyl, or C₁-C₃ haloalkoxy;
R₁ is selected from allyl, propargyl, cyclopropylmethyl, CNCH₂—, CNCH₂CH₂—, CNCH₂CH₂CH₂—, CNCH₂CH₂CH₂CH₂—, or

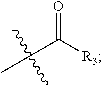

R₂ is selected from hydrogen or cyanide; and
R₃ is selected from methyl, ethyl, n-propyl, isopropyl, n-butyl, isobutyl, sec-butyl, tert-butyl, cyclopropyl, cyclobutyl, cyclopentyl, cyclohexyl, CH₃OCH₂—, CH₃CH₂OCH₂—, CH₃CH₂CH₂OCH₂—, (CH₃)₂CHOCH₂—, CH₃CH₂CH₂CH₂OCH₂—, (CH₃)₃COCH₂—, CH₃OCH₂CH₂—, CH₃CH₂OCH₂CH₂—, CH₃CH₂CH₂OCH₂CH₂—, or CH₃CH₂CH₂CH₂OCH₂CH₂—;
where the phenylpyrazole compound is a stereoisomer of the compound as shown in the general formula I; a salt of the compound as shown in the general formula I; or a salt of the stereoisomer of the compound as shown in the general formula I.

In a possible implementation, in the general formula I,
W₁ is selected from fluorine, trifluoromethyl, or pentafluoroethyl;
W₂ is selected from fluorine or trifluoromethyl;
Y₁ is selected from chlorine or bromine;
Y₂ is selected from chlorine, bromine, trifluoromethyl, difluoromethoxy, or trifluoromethoxy;
R₁ is selected from allyl, propargyl, cyclopropylmethyl, CNCH₂—, CNCH₂CH₂—, CNCH₂CH₂CH₂—, CNCH₂CH₂CH₂CH₂—, or

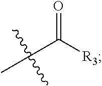

R₂ is selected from hydrogen or cyanide; and
R₃ is selected from methyl, ethyl, n-propyl, isopropyl, cyclopropyl, CH₃OCH₂—, CH₃CH₂OCH₂—, or CH₃CH₂CH₂OCH₂—;
where the phenylpyrazole compound is a stereoisomer of the compound as shown in the general formula I; a salt of the compound as shown in the general formula I; or a salt of the stereoisomer of the compound as shown in the general formula I.

In a possible implementation, when W₁ and W₂ are selected from trifluoromethyl and R₁ is selected from allyl, propargyl, cyclopropylmethyl, CNCH₂—, CNCH₂CH₂—, CNCH₂CH₂CH₂—, or CNCH₂CH₂CH₂CH₂—, the phenylpyrazole compound is selected from compounds in Table 1, where the compounds in the Table 1 have the structure as shown in the general formula I, and Y₁, Y₂, R₁, and R₂ are as shown in the Table 1:

TABLE 1

| Compound | | | | |
|---|---|---|---|---|
| Number | Y₁ | Y₂ | R₁ | R₂ |
| 1 | Cl | Cl | Allyl | H |
| 2 | Cl | Cl | Propargyl | H |
| 3 | Cl | Cl | Cyclopropylmethyl | H |
| 4 | Cl | Cl | CNCH₂— | H |
| 5 | Cl | Cl | CNCH₂CH₂— | H |
| 6 | Cl | Cl | CNCH₂CH₂CH₂— | H |
| 7 | Cl | Cl | CNCH₂CH₂CH₂CH₂— | H |
| 8 | Cl | Cl | Allyl | CN |
| 9 | Cl | Cl | Propargyl | CN |
| 10 | Cl | Cl | Cyclopropylmethyl | CN |
| 11 | Cl | Cl | CNCH₂— | CN |
| 12 | Cl | Cl | CNCH₂CH₂— | CN |
| 13 | Cl | Cl | CNCH₂CH₂CH₂— | CN |
| 14 | Cl | Cl | CNCH₂CH₂CH₂CH₂— | CN |
| 15 | Br | Br | Allyl | H |
| 16 | Br | Br | Propargyl | H |
| 17 | Br | Br | Cyclopropylmethyl | H |
| 18 | Br | Br | CNCH₂— | H |
| 19 | Br | Br | CNCH₂CH₂— | H |
| 20 | Br | Br | CNCH₂CH₂CH₂— | H |
| 21 | Br | Br | CNCH₂CH₂CH₂CH₂— | H |
| 22 | Br | Br | Allyl | CN |
| 23 | Br | Br | Propargyl | CN |
| 24 | Br | Br | Cyclopropylmethyl | CN |
| 25 | Br | Br | CNCH₂— | CN |
| 26 | Br | Br | CNCH₂CH₂— | CN |
| 27 | Br | Br | CNCH₂CH₂CH₂— | CN |
| 28 | Br | Br | CNCH₂CH₂CH₂CH₂— | CN |
| 29 | Br | Cl | Allyl | H |
| 30 | Br | Cl | Propargyl | H |
| 31 | Br | Cl | Cyclopropylmethyl | H |
| 32 | Br | Cl | CNCH₂— | H |
| 33 | Br | Cl | CNCH₂CH₂— | H |
| 34 | Br | Cl | CNCH₂CH₂CH₂— | H |
| 35 | Br | Cl | CNCH₂CH₂CH₂CH₂— | H |
| 36 | Br | Cl | Allyl | CN |
| 37 | Br | Cl | Propargyl | CN |
| 38 | Br | Cl | Cyclopropylmethyl | CN |
| 39 | Br | Cl | CNCH₂— | CN |
| 40 | Br | Cl | CNCH₂CH₂— | CN |
| 41 | Br | Cl | CNCH₂CH₂CH₂— | CN |
| 42 | Br | Cl | CNCH₂CH₂CH₂CH₂— | CN |
| 43 | Br | CF₃ | Allyl | H |
| 44 | Br | CF | Propargyl | H |
| 45 | Br | CF₃ | Cyclopropylmethyl | H |
| 46 | Br | CF₃ | CNCH₂— | H |
| 47 | Br | CF₃ | CNCH₂CH₂— | H |
| 48 | Br | CF₃ | CNCH₂CH₂CH₂— | H |
| 49 | Br | CF₃ | CNCH₂CH₂CH₂CH₂— | H |
| 50 | Br | CF₃ | Allyl | CN |
| 51 | Br | CF₃ | Propargyl | CN |
| 52 | Br | CF₃ | Cyclopropylmethyl | CN |
| 53 | Br | CF₃ | CNCH₂— | CN |
| 54 | Br | CF₃ | CNCH₂CH₂— | CN |
| 55 | Br | CF₃ | CNCH₂CH₂CH₂— | CN |
| 56 | Br | CF₃ | CNCH₂CH₂CH₂CH₂— | CN |
| 57 | Cl | CF₃ | Allyl | H |
| 58 | Cl | CF₃ | Propargyl | H |
| 59 | Cl | CF₃ | Cyclopropylmethyl | H |
| 60 | Cl | CF₃ | CNCH₂— | H |
| 61 | Cl | CF₃ | CNCH₂CH₂— | H |
| 62 | Cl | CF₃ | CNCH₂CH₂CH₂— | H |
| 63 | Cl | CF₃ | CNCH₂CH₂CH₂CH₂— | H |
| 64 | Cl | CF₃ | Allyl | CN |
| 65 | Cl | CF₃ | Propargyl | CN |
| 66 | Cl | CF₃ | Cyclopropylmethyl | CN |
| 67 | Cl | CF₃ | CNCH₂— | CN |
| 68 | Cl | CF₃ | CNCH₂CH₂— | CN |
| 69 | Cl | CF₃ | CNCH₂CH₂CH₂— | CN |
| 70 | Cl | CF₃ | CNCH₂CH₂CH₂CH₂— | CN |
| 71 | Br | OCF₃ | Allyl | H |
| 72 | Br | OCF₃ | Propargyl | H |
| 73 | Br | OCF₃ | Cyclopropylmethyl | H |
| 74 | Br | OCF₃ | CNCH₂— | H |
| 75 | Br | OCF₃ | CNCH₂CH₂— | H |
| 76 | Br | OCF₃ | CNCH₂CH₂CH₂— | H |
| 77 | Br | OCF₃ | CNCH₂CH₂CH₂CH₂— | H |
| 78 | Br | OCF₃ | Allyl | CN |
| 79 | Br | OCF₃ | Propargyl | CN |
| 80 | Br | OCF₃ | Cyclopropylmethyl | CN |
| 81 | Br | OCF₃ | CNCH₂— | CN |
| 82 | Br | OCF₃ | CNCH₂CH₂— | CN |
| 83 | Br | OCF₃ | CNCH₂CH₂CH₂— | CN |
| 84 | Br | OCF₃ | CNCH₂CH₂CH₂CH₂— | CN |
| 85 | Cl | OCF₃ | Allyl | H |
| 86 | Cl | OCF₃ | Propargyl | H |
| 87 | Cl | OCF₃ | Cyclopropylmethyl | H |
| 88 | Cl | OCF₃ | CNCH₂— | H |
| 89 | Cl | OCF₃ | CNCH₂CH₂— | H |
| 90 | Cl | OCF₃ | CNCH₂CH₂CH₂— | H |
| 91 | Cl | OCF₃ | CNCH₂CH₂CH₂CH₂— | H |
| 92 | Cl | OCF₃ | Allyl | CN |
| 93 | Cl | OCF₃ | Propargyl | CN |
| 94 | Cl | OCF₃ | Cyclopropylmethyl | CN |
| 95 | Cl | OCF₃ | CNCH₂— | CN |
| 96 | Cl | OCF₃ | CNCH₂CH₂— | CN |
| 97 | Cl | OCF₃ | CNCH₂CH₂CH₂— | CN |
| 98 | Cl | OCF₃ | CNCH₂CH₂CH₂CH₂— | CN. |

In a possible implementation, when W₁ and W₂ are selected from trifluoromethyl and R₁ is selected from

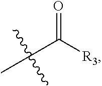

the phenylpyrazole compound is selected from compounds in Table 2, where the compounds in the Table 2 have the structure as shown in the general formula I, and Y₁, Y₂, R₂, and R₃ are as shown in the Table 2:

TABLE 2

| Compound | | | | |
|---|---|---|---|---|
| Number | Y₁ | Y₂ | R₂ | R₃ |
| 99 | Cl | Cl | H | Methyl |
| 100 | Cl | Cl | H | Ethyl |
| 101 | Cl | Cl | H | N-propyl |
| 102 | Cl | Cl | H | Isopropyl |
| 103 | Cl | Cl | H | N-butyl |
| 104 | Cl | Cl | H | Cyclopropyl |
| 105 | Cl | Cl | H | CH₃OCH₂— |
| 106 | Cl | Cl | H | CH₃CH₂OCH₂— |
| 107 | Cl | Cl | H | CH₃CH₂CH₂OCH₂— |
| 108 | Cl | Cl | CN | Methyl |
| 109 | Cl | Cl | CN | Ethyl |
| 110 | Cl | Cl | CN | N-propyl |
| 111 | Cl | Cl | CN | Isopropyl |
| 112 | Cl | Cl | CN | N-butyl |
| 113 | Cl | Cl | CN | Cyclopropyl |
| 114 | Cl | Cl | CN | CH₃OCH₂— |
| 115 | Cl | Cl | CN | CH₃CH₂OCH₂— |
| 116 | Cl | Cl | CN | CH₃CH₂CH₂OCH₂— |
| 117 | Br | Br | H | Methyl |
| 118 | Br | Br | H | Ethyl |
| 119 | Br | Br | H | N-propyl |
| 120 | Br | Br | H | Isopropyl |
| 121 | Br | Br | H | N-butyl |
| 122 | Br | Br | H | Cyclopropyl |
| 123 | Br | Br | H | CH₃OCH₂— |
| 124 | Br | Br | H | CH₃CH₂OCH₂— |
| 125 | Br | Br | H | CH₃CH₂CH₂OCH₂— |
| 126 | Br | Br | CN | Methyl |
| 127 | Br | Br | CN | Ethyl |
| 128 | Br | Br | CN | N-propyl |
| 129 | Br | Br | CN | Isopropyl |
| 130 | Br | Br | CN | N-butyl |
| 131 | Br | Br | CN | Cyclopropyl |
| 132 | Br | Br | CN | CH₃OCH₂— |
| 133 | Br | Br | CN | CH₃CH₂OCH₂— |
| 134 | Br | Br | CN | CH₃CH₂CH₂OCH₂— |
| 135 | Br | Cl | H | Methyl |
| 136 | Br | Cl | H | Ethyl |
| 137 | Br | Cl | H | N-propyl |
| 138 | Br | Cl | H | Isopropyl |
| 139 | Br | Cl | H | N-butyl |
| 140 | Br | Cl | H | Cyclopropyl |
| 141 | Br | Cl | H | CH₃OCH₂— |
| 142 | Br | Cl | H | CH₃CH₂OCH₂— |
| 143 | Br | Cl | H | CH₃CH₂CH₂OCH₂— |
| 144 | Br | Cl | CN | Methyl |
| 145 | Br | Cl | CN | Ethyl |
| 146 | Br | Cl | CN | N-propyl |
| 147 | Br | Cl | CN | Isopropyl |
| 148 | Br | Cl | CN | N-butyl |
| 149 | Br | Cl | CN | Cyclopropyl |
| 150 | Br | Cl | CN | CH₃OCH₂— |
| 151 | Br | Cl | CN | CH₃CH₂OCH₂— |
| 152 | Br | Cl | CN | CH₃CH₂CH₂OCH₂— |
| 153 | Br | CF₃ | H | Methyl |
| 154 | Br | CF₃ | H | Ethyl |
| 155 | Br | CF₃ | H | N-propyl |
| 156 | Br | CF₃ | H | Isopropyl |
| 157 | Br | CF₃ | H | N-butyl |
| 158 | Br | CF₃ | H | Cyclopropyl |
| 159 | Br | CF₃ | H | CH₃OCH₂— |
| 160 | Br | CF₃ | H | CH₃CH₂OCH₂— |
| 161 | Br | CF₃ | H | CH₃CH₂CH₂OCH₂— |
| 162 | Br | CF₃ | CN | Methyl |
| 163 | Br | CF₃ | CN | Ethyl |
| 164 | Br | CF₃ | CN | N-propyl |
| 165 | Br | CF₃ | CN | Isopropyl |
| 166 | Br | CF₃ | CN | N-butyl |
| 167 | Br | CF₃ | CN | Cyclopropyl |
| 168 | Br | CF₃ | CN | CH₃OCH₂— |
| 169 | Br | CF₃ | CN | CH₃CH₂OCH₂— |
| 170 | Br | CF₃ | CN | CH₃CH₂CH₂OCH₂— |
| 171 | Cl | CF₃ | H | Methyl |
| 172 | Cl | CF₃ | H | Ethyl |
| 173 | Cl | CF₃ | H | N-propyl |
| 174 | Cl | CF₃ | H | Isopropyl |
| 175 | Cl | CF₃ | H | N-butyl |
| 176 | Cl | CF₃ | H | Cyclopropyl |
| 177 | Cl | CF₃ | H | CH₃OCH₂— |
| 178 | Cl | CF₃ | H | CH₃CH₂OCH₂— |
| 179 | Cl | CF₃ | H | CH₃CH₂CH₂OCH₂— |
| 180 | Cl | CF₃ | CN | Methyl |
| 181 | Cl | CF₃ | CN | Ethyl |
| 182 | Cl | CF₃ | CN | N-propyl |
| 183 | Cl | CF₃ | CN | Isopropyl |
| 184 | Cl | CF₃ | CN | N-butyl |
| 185 | Cl | CF₃ | CN | Cyclopropyl |
| 186 | Cl | CF₃ | CN | CH₃OCH₂— |
| 187 | Cl | CF₃ | CN | CH₃CH₂OCH₂— |
| 188 | Cl | CF₃ | CN | CH₃CH₂CH₂OCH₂— |
| 189 | Br | OCF₃ | H | Methyl |
| 190 | Br | OCF₃ | H | Ethyl |
| 191 | Br | OCF₃ | H | N-propyl |
| 192 | Br | OCF₃ | H | Isopropyl |
| 193 | Br | OCF₃ | H | N-butyl |
| 194 | Br | OCF₃ | H | Cyclopropyl |
| 195 | Br | OCF₃ | H | CH₃OCH₂— |
| 196 | Br | OCF₃ | H | CH₃CH₂OCH₂— |
| 197 | Br | OCF₃ | H | CH₃CH₂CH₂OCH₂— |
| 198 | Br | OCF₃ | CN | Methyl |
| 199 | Br | OCF₃ | CN | Ethyl |
| 200 | Br | OCF₃ | CN | N-propyl |
| 201 | Br | OCF₃ | CN | Isopropyl |
| 202 | Br | OCF₃ | CN | N-butyl |
| 203 | Br | OCF₃ | CN | Cyclopropyl |
| 204 | Br | OCF₃ | CN | CH₃OCH₂— |
| 205 | Br | OCF₃ | CN | CH₃CH₂OCH₂— |
| 206 | Br | OCF₃ | CN | CH₃CH₂CH₂OCH₂— |
| 207 | Cl | OCF₃ | H | Methyl |
| 208 | Cl | OCF₃ | H | Ethyl |
| 209 | Cl | OCF₃ | H | N-propyl |
| 210 | Cl | OCF₃ | H | Isopropyl |
| 211 | Cl | OCF₃ | H | N-butyl |
| 212 | Cl | OCF₃ | H | Cyclopropyl |
| 213 | Cl | OCF₃ | H | CH₃OCH₂— |
| 214 | Cl | OCF₃ | H | CH₃CH₂OCH₂— |
| 215 | Cl | OCF₃ | H | CH₃CH₂CH₂OCH₂— |
| 216 | Cl | OCF₃ | CN | Methyl |
| 217 | Cl | OCF₃ | CN | Ethyl |
| 218 | Cl | OCF₃ | CN | N-propyl |
| 219 | Cl | OCF₃ | CN | Isopropyl |
| 220 | Cl | OCF₃ | CN | N-butyl |
| 221 | Cl | OCF₃ | CN | Cyclopropyl |
| 222 | Cl | OCF₃ | CN | CH₃OCH₂— |
| 223 | Cl | OCF₃ | CN | CH₃CH₂OCH₂— |
| 224 | Cl | OCF₃ | CN | CH₃CH₂CH₂OCH₂—. |

In a possible implementation, when W₁ and W₂ are selected from trifluoromethyl and R₁ is selected from allyl, propargyl, cyclopropylmethyl, CNCH₂—, CNCH₂CH₂—, CNCH₂CH₂CH₂—, or CNCH₂CH₂CH₂CH₂— the phenylpyrazole compound is selected from compounds in Table 3, where the compounds in the Table 3 have the structure as shown in the general formula I, and Y₁, Y₂, R₁, and R₂ are as shown in the Table 3:

TABLE 3

| Compound | | | | |
|---|---|---|---|---|
| Number | Y₁ | Y₂ | R₁ | R₂ |
| 1 | Cl | Cl | Allyl | H |
| 2 | Cl | Cl | Propargyl | H |
| 3 | Cl | Cl | Cyclopropylmethyl | H |
| 4 | Cl | Cl | CNCH₂— | H |
| 5 | Cl | Cl | CNCH₂CH₂— | H |
| 11 | Cl | Cl | CNCH₂— | CN |
| 12 | Cl | Cl | CNCH₂CH₂— | CN |
| 18 | Br | Br | CNCH₂— | H |
| 19 | Br | Br | CNCH₂CH₂— | H |
| 25 | Br | Br | CNCH₂— | CN |
| 26 | Br | Br | CNCH₂CH₂— | CN |
| 32 | Br | Cl | CNCH₂— | H |
| 33 | Br | Cl | CNCH₂CH₂— | H |
| 34 | Br | Cl | CNCH₂CH₂CH₂— | H |
| 39 | Br | Cl | CNCH₂— | CN |
| 40 | Br | Cl | CNCH₂CH₂— | CN |
| 46 | Br | CF₃ | CNCH₂— | H |
| 47 | Br | CF₃ | CNCH₂CH₂— | H |
| 53 | Br | CF₃ | CNCH₂— | CN |
| 54 | Br | CF₃ | CNCH₂CH₂— | CN |
| 60 | Cl | CF₃ | CNCH₂— | H |
| 61 | Cl | CF₃ | CNCH₂CH₂— | H |
| 67 | Cl | CF₃ | CNCH₂— | CN |
| 68 | Cl | CF₃ | CNCH₂CH₂— | CN |
| 74 | Br | OCF₃ | CNCH₂— | H |
| 75 | Br | OCF₃ | CNCH₂CH₂— | H |
| 81 | Br | OCF₃ | CNCH₂— | CN |
| 82 | Br | OCF₃ | CNCH₂CH₂— | CN |
| 88 | Cl | OCF₃ | CNCH₂— | H |
| 89 | Cl | OCF₃ | CNCH₂CH₂— | H |
| 95 | Cl | OCF₃ | CNCH₂— | CN |
| 96 | Cl | OCF₃ | CNCH₂CH₂— | CN. |

In a possible implementation, when W₁ and W₂ are selected from trifluoromethyl and R₁ is selected from

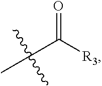

the phenylpyrazole compound is selected from compounds in Table 4, where the compounds in the Table 4 have the structure as shown in the general formula I, and Y₁, Y₂, R₂, and R₃ are as shown in the Table 4:

TABLE 4

| Compound | | | | |
|---|---|---|---|---|
| Number | Y₁ | Y₂ | R₂ | R₃ |
| 99 | Cl | Cl | H | Methyl |
| 100 | Cl | Cl | H | Ethyl |
| 101 | Cl | Cl | H | N-propyl |
| 103 | Cl | Cl | H | N-butyl |
| 104 | Cl | Cl | H | Cyclopropyl |
| 105 | Cl | Cl | H | CH₃OCH₂— |
| 106 | Cl | Cl | H | CH₃CH₂OCH₂— |
| 107 | Cl | Cl | H | CH₃CH₂CH₂OCH₂— |
| 108 | Cl | Cl | CN | Methyl |
| 109 | Cl | Cl | CN | Ethyl |
| 110 | Cl | Cl | CN | N-propyl |
| 112 | Cl | Cl | CN | N-butyl |
| 113 | Cl | Cl | CN | Cyclopropyl |
| 114 | Cl | Cl | CN | CH₃OCH₂— |
| 115 | Cl | Cl | CN | CH₃CH₂OCH₂— |
| 116 | Cl | Cl | CN | CH₃CH₂CH₂OCH₂—. |

In a possible implementation, the salt of the compound as shown in the general formula I includes: a salt formed by the compound as shown in the general formula I and hydrochloric acid, hydrobromic acid, sulfuric acid, phosphoric acid, formic acid, acetic acid, propionic acid, butyric acid, valeric acid, trifluoroacetic acid, oxalic acid, propanedioic acid, methanesulfonic acid, 4-toluenesulfonic acid, malic acid, fumaric acid, lactic acid, maleic acid, salicylic acid, tartaric acid, or citric acid.

In a possible implementation, the salt of the stereoisomer of the compound as shown in the general formula I includes: a salt formed by the stereoisomer of the compound as shown in the general formula I and hydrochloric acid, hydrobromic acid, sulfuric acid, phosphoric acid, formic acid, acetic acid, propionic acid, butyric acid, valeric acid, trifluoroacetic acid, oxalic acid, propanedioic acid, methanesulfonic acid, 4-toluenesulfonic acid, malic acid, fumaric acid, lactic acid, maleic acid, salicylic acid, tartaric acid, or citric acid.

An embodiment of the present disclosure further provides a preparation method of the phenylpyrazole compound, as follows (various groups in formulas are defined the same as above unless otherwise specified, and in the formulas, LG=Cl, Br, or I):

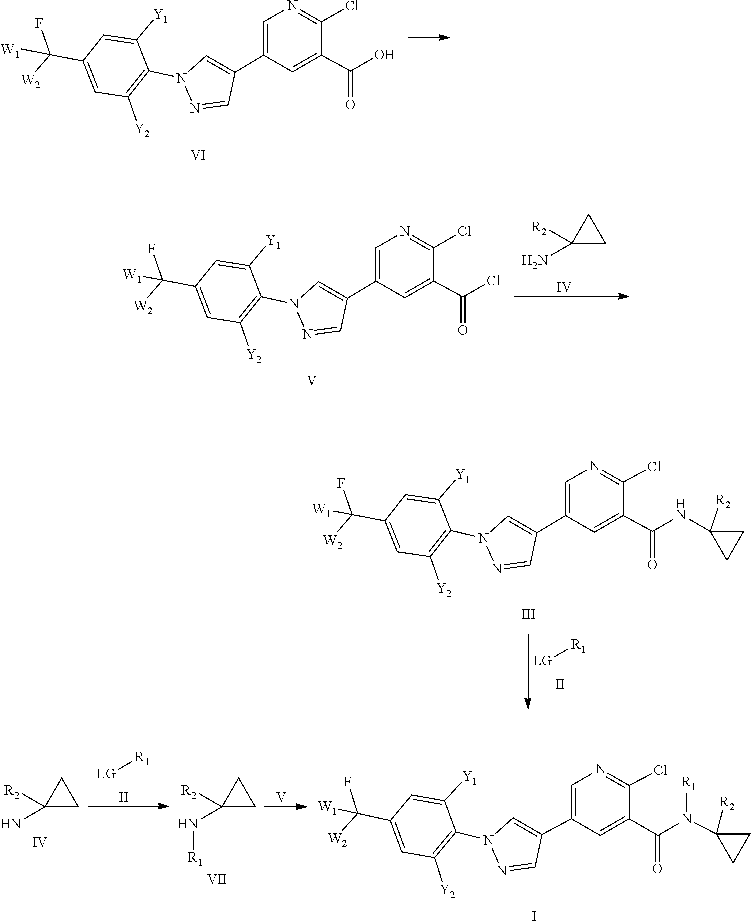

A compound as shown in general formula V is prepared by a reaction of a compound as shown in general formula VI with an acyl chlorination reagent using a conventional method.

In a possible implementation, the acyl chlorination reagent includes: one or more of thionyl chloride, oxaloyl chloride, phosgene, phosphorus oxychloride, phosphorus pentachloride, phosphorus trichloride, or triphosgene.

Route 1: The compound as shown in the general formula V and a compound as shown in general formula IV can be reacted in a suitable solvent at the temperature from −10° C. to a boiling point of the solvent for 0.5-48 h to obtain a compound as shown in general formula III; and the compound as shown in the general formula III and a compound as shown in general formula II (an alkylation agent or an acylation agent) can be reacted in a suitable solvent at the temperature from −10° C. to a boiling point of the solvent for 0.5-48 h to obtain the compound as shown in general formula I.

Route 2: The compound as shown in the general formula IV and the compound as shown in the general formula II can be reacted in a suitable solvent at the temperature from −10° C. to a boiling point of the solvent for 0.5-48 h to obtain a compound as shown in general formula VII; and the compound as shown in the general formula VII and the compound as shown in the general formula V can be reacted in a suitable solvent at the temperature from −10° C. to a boiling point of the solvent for 0.5-48 h to obtain the compound as shown in the general formula I.

In a possible implementation, the solvents of the reactions involved in Route 1 and Route 2 include: one or more of aromatic hydrocarbons, such as benzene, toluene, and xylene, etc.; ketones, such as acetone, methyl ethyl ketone, and methyl isobutyl ketone, etc.; haloalkanes, such as chloroform and dichloromethane, etc.; esters, such as methyl acetate and ethyl acetate, etc.; ethers, such as tetrahydrofuran, dioxane, diethyl ether, and 1,2-dimethoxyethane, etc.; and polar solvents, such as water, acetonitrile, N, N-dimethylformamide, N-methylpyrrolidone, and dimethyl sulfoxide, etc.

In a possible implementation, the reactions involved in Route 1 and Route 2 may be carried out in the presence of an base, and the base includes: one or more of organic bases, such as trimethylamine, triethylamine, pyridine, DBU, 4-dimethylaminopyridine, N,N-diisopropylmethylamine, and N,N-diisopropylethylamine, etc.; alkali metal hydrides, such as sodium hydride and potassium hydride, etc.; alkali metal hydroxides, such as sodium hydroxide and potassium hydroxide, etc.; alkaline-earth metal hydroxides, such as calcium hydroxide, etc.; alkali metal carbonates, such as sodium carbonate and potassium carbonate, etc.; alkali metal bicarbonates, such as sodium bicarbonate, etc.; and metal alkoxides, such as sodium methoxide, sodium ethoxide, potassium ethoxide, potassium tert-butoxide, and sodium tert-butoxide, etc.

The intermediate compound as shown in the general formula VI can be prepared by a well-known method, such as a method reported in WO2019243243, WO2015067646, WO2015067647, or WO2018104214. The compound as shown in the general formula II and the compound as shown in the general formula IV are usually commercially available and can also be prepared by conventional methods.

An embodiment of the present disclosure further provides use of the phenylpyrazole compound in preparation of an insecticide and/or an acaricide.

In a possible implementation, the insecticide is used for preventing and controlling one or more of the following insects:
beetles (Coleopteran insects), for example, Callosobruchus Chinensis, Sitophilus zeamais, Tribolium Castaneum, Epilachna vigintioctomaculata, Agriotes ogurae fuscicollis, Anomala rufocuprea, Leptinotarsa decemlineata, Diabrotica spp., Monochamus alternatus endai, Lissorhoptrus oryzophilus, or Lyctus bruneus;

Lepidopteran pests, for example, Lymantria dispar, Malacosoma neustria, Pieris rapae crucivora, Spodoptera litura, Mamestra brassicae, Chilo suppressalis, Ostrinia nubilalis, Cadra cautella, Adoxophyes honmai, Cydia pomonella, Agrotis segetum, Galleria mellonella, Plutella xylostella, Heliothis virescens, or Phyllocnistis citrella;

Hemipterous pests, for example, Nephotettix cincticeps, Nilaparvata lugens, Pseudococcus comstocki, Unaspis yanonensis, Myzus persicas, Aphis pomi, Aphis gossypii, Lipaphis erysimi, Stephanitis nashi, Nezara spp., Trialeurodes vaporariorum, or Pshylla spp.; Thysanoptera pests, for example, Thrips palmi, or Franklinella occidentalis; Orthopteran pests, for example, Gryllotalpa Africana, or Locusta migratoria; Blattarian pests, for example, Blattella germanica, Periplaneta americana, Reticulitermes speratus, or Coptotermes formosanus; and Dipterous pests, for example, Musca domestica, Aedesaegypti, Delia platura, Culex pipiens pallens, Anopheles sinensis, Culex tritaeniorhynchus, Liriomyza trifolii, etc.

In a possible implementation, the acaricide is used for preventing and controlling one or more of the following acarids: Tetranychus cinnabarinus, Tetrahychus urticae, Panonychus citri, Aculops pelekassi, Tarsonemus spp., etc.

In a possible implementation, the insecticide and/or the acaricide is used for preventing and controlling one or more of Mythimna separata, Plutella xylostella, and Tetranychus cinnabarinus.

An embodiment of the present disclosure further provides an insecticide or acaricide formulation. The insecticide or acaricide formulation includes the phenylpyrazole compound as an active component, and further includes one or more excipients.

In a possible implementation, the insecticide or acaricide formulation is selected from the following dosage forms: solution, emulsion, wettable powder, granular wettable powder, suspension concentrate, powder, foaming agent, ointment, tablet, granule, aerosol, natural reagent impregnated with an active compound, synthetic reagent impregnated with an active compound, microcapsule, seed coating agent, formulation equipped with a combustion device (the combustion device may be a smoke tube, a mist tube, a tank, and a coil pipe, etc.), and ULV (cold fogging concentrate or hot fogging concentrate), etc. The insecticide formulation, acaricide formulation, or an animal parasite control agent may be prepared by a known method, for example, may be prepared by mixing the active component with a filler (e.g.: a liquid diluent or carrier, a liquefied gas diluent or carrier, or a solid diluent or carrier) and optionally mixing with a surfactant (i.e., an emulsifier and/or a dispersant and/or a blowing agent), etc.

In a possible implementation, the excipients include one or more of the following: a filler (e.g., a liquid diluent or carrier, a liquefied gas diluent or carrier, or a solid diluent or carrier), a surfactant (e.g., an emulsifier and/or a dispersant and/or a blowing agent), an adhesive, and a colorant;

The liquid diluent or carrier may include, for example, aromatic hydrocarbons (xylene, toluene, alkyl naphthalene, etc.), chlorinated aromatic hydrocarbons or chlorinated aliphatic hydrocarbons (e.g., chlorobenzene, vinyl chloride, dichloromethane, etc.), aliphatic hydrocarbons (e.g., cyclohexane or paraffin (e.g., mineral oil fraction)), alcohols (e.g., butanol, ethylene glycol, and ethers or esters thereof, etc.), ketones (e.g., acetone, methyl ethyl ketone, methyl isobutyl ketone, cyclohexanone, etc.), strong polar solvents (e.g., dimethylformamide, or dimethyl sulfoxide), or water, etc. When water is used as a filler, for example, an organic solvent may be used as a co-solvent;

The liquefied gas diluent or carrier may include those present in the form of gas at atmospheric pressure and temperature, for example, propane, nitrogen, carbon dioxide, and aerosol propellants such as haloalkanes;

The solid diluent may include crushed natural minerals (e.g., kaolin, clay, talc, chalk, quartz, attapulgite, montmorillonite, or diatomite, etc.) and crushed synthetic minerals (e.g., finely dispersed silicic acid, alumina, and silicates, etc.);

The emulsifier and/or the blowing agent may include non-ionic and anionic emulsifiers [e.g., polyoxyethylene fatty acid esters, polyoxyethylene fatty acid alcohol ethers (e.g., alkyl aryl polyethylene glycol ethers), alkyl sulfonates, alkyl sulfates, and aryl sulfonates] and albumin hydrolysates, etc;

The dispersant may include lignin sulfite waste liquid and methyl cellulose;

The adhesive may include carboxymethyl cellulose and natural or synthetic polymers (e.g., arabic gum, polyvinyl alcohol, and polyvinyl acetate, etc.).

The colorant may include inorganic pigments (e.g., iron oxide, titanium oxide, and Prussian blue, etc.); organic dyes, such as alizarin dyes, azo dyes, or metal phthalocyanine dyes; and trace elements, such as iron salts, manganese salts, boron salts, copper salts, cobalt salts, molybdenum salts, or zinc salts.

In addition, the phenylpyrazole compound of the present disclosure may exist as a mixture with a synergist, and the synergist itself does not need to be active. More precisely, the synergist is a compound for enhancing the activity of the active compound.

In a possible implementation, an amount of the phenylpyrazole compound contained in the insecticide or acaricide formulation is 0.1 to 99 wt %, optionally 0.5 to 90 wt %.

An embodiment of the present disclosure further provides an insecticide or acaricide composition. The insecticide or acaricide composition includes a mixture of the phenylpyrazole compound and other active compounds (e.g., an insecticide, a poison bait, a disinfectant, an acaricide, a nematicide, a fungicide, a growth regulator, a herbicide, etc.). The mixture may be provided in the form of a bulk drug and may also be provided in the form of a commercially available formulation or in a use form prepared by the formulation thereof.

An embodiment of the present disclosure further provides a method for controlling agricultural or forestry pests and/or acarids. The method includes the following step: administering an effective dose of a material to the pests, the acarids, or a growth medium thereof to be controlled, where the material is selected from one or more of the following groups: the phenylpyrazole compound, the insecticide or acaricide formulation, and the insecticide or acaricide composition.

An embodiment of the present disclosure further provides use of the phenylpyrazole compound in preparation of an animal parasite control agent. In the field of veterinary medicine, that is, veterinary science, the phenylpyrazole compound of the present disclosure can be used for effectively resisting a variety of harmful animal parasites, especially endoparasites and ectoparasites.

In a possible implementation, the animal parasites include one or more of the following:
Anoplurida, for example, Haematopinus spp., Linognathus spp., Pediculus spp., Phtirus spp., and Solenopotes spp.; specifically, representative examples including Linognathus setosus and Solenopotes capillatus;
Mallopha (Linognathus vituli, Linognathus ovillus, Linognathus oviformis, Linognathus pedalis, Linognathus stenopsis, Haematopinus asini macrocephalus, Haematopinus eurysternus, Haematopinus suis, Pediculus humanus capitis, Pediculus humanus corporis, Phylloera vastatrix, Phthirus pubis) gida, Amblycerina, and Ischnocerin, for example, Trimenopon spp., Menopon spp., Trinoton spp., Bovicola spp., Werneckiella spp., Lepikentron spp., Damalina spp., Trichodectes spp., and Felicola spp.; specifically, representative examples including Bovicola bovis, Bovicola ovis, Bovicola limbata, Damalina bovis, Trichodectes canis, Felicola subrostratus, Bovicola caprae, Lepikentron ovis, and Werneckiella equi;
Diptera as well as Nematocerina and Brachycerina thereof, for example, Aedes spp., Anopheles spp., Culex spp., Simulium spp., Eusimulium spp., Phlebotomus spp., Lutzomyia spp., Culicoides spp., Chrysops spp., Odagmia spp., Wilhelmia spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp., Rhinoestrus spp., and Tipula spp.; specifically, representative examples including Aedes aegypti, Aedes albopictus, Aedes taeniorhynchus, Anopheles gambiae, Anopheles maculipennis, Calliphora erythrocephala, Chrysozona pluvialis, Culex quinquefasciatus, Culex pipiens, Culex tarsalis, Fannia canicularis, Sarcophaga carnaria, Stomoxys calcitrans, Tipula paludosa, Lucilia cuprina, Lucilia sericata, Simulium reptans, Phlebotomus papatasi, Phlebotomus longipalpis, Odagmia ornata, Wilhelmia equina, Boophthora erythrocephala, Tabanus bromius, Tabanus spodopterus, Tabanus atratus, Tabanus sudeticus, Hybomitra ciurea, Chrysops caecutiens, Chrysops relictus, Haematopota pluvialis, Haematopota italica, Musca autumnalis, Musca domestica, Haematobia irritans irritans, Haematobia irritans exigua, Haematobia stimulans, Hydrotaea irritans, Hydrotaea albipuncta, Chrysomya chloropyga, Chrysomya bezziana, Oestrus ovis, Hypoderma bovis, Hypoderma lineatum, Przhevalskiana silenus, Dermatobia hominis, Melophagus ovinus, Lipoptena capreoli, Lipoptena cervi, Hippobosca variegata, Hippobosca equina, Gasterophilus intestinalis, Gasterophilus haemorroidalis, Gasterophilus interrnis, Gasterophilus nasalis, Gasterophilus nigricornis, Gasterophilus pecorum, and Braula coeca;
Siphonapterida, for example, Pulex spp., Ctenocephalides spp., Tunga spp., Xenopsylla spp., and Ceratophyllus spp.; specifically, representative examples including Ctenocephalides canis, Ctenocephalides felis, Pulex irritans, Tunga penetrans, and Xenopsylla cheopis;
Heteropterida, for example, Cimex spp., Triatoma spp., Rhodnius spp., and Panstrongylus spp.;
Blattarida, for example, Blatta orientalis, Periplaneta americana, Blattela germanica, and Supella spp. (e.g., Suppella longipalpa);
Acari (or Acarina), Metastigmata, and Mesostigmata, for example, Argas spp., Ornithodorus spp., Otobius spp., Ixodes spp., Amblyomma spp., Rhipicephalus (Boophilus) spp., Dermacentor spp., Haemophysalis spp., Hyalomma spp., Dermanyssus spp., Rhipicephalus spp. (original genus of heteroparasitic acarids), Ornithonyssus spp., Raillietia spp., Pneumonyssus spp., Sternostoma spp., Varroa spp., and Acarapis spp.; specifically, representative examples including Argas persicus, Argas reflexus, Ornithodorus moubata, Otobius megnini, Rhipicephalus (Boophilus) microplus, Rhipicephalus (Boophilus) decoloratus, Rhipicephalus (Boophilus) annulatus, Rhipicephalus (Boophilus) calceratus, Hyalomma anatolicum, Hyalomma aegypticum, Hyalomma marginatum, Hyalomma transiens, Rhipicephalus evertsi, Ixodes ricinus, Ixodes hexagonus, Ixodes canisuga, Ixodes pilosus, Ixodes rubicundus, Ixodes scapularis, Ixodes holocyclus, Haemaphysalis concinna, Haemaphysalis punctata, Haemaphysalis cinnabarina, Haemaphysalis otophila, Haemaphysalis leachi, Haemaphysalis longicorni, Dermacentor marginatus, Dermacentor reticulatus, Dermacentor pictus, Dermacentor albipictus, Dermacentor andersoni, Dermacentor variabilis, Hyalomma mauritanicum, Rhipicephalus sanguineus, Rhipicephalus bursa, Rhipicephalus appendiculatus, Rhipicephalus capensis, Rhipicephalus turanicus, Rhipicephalus zambeziensis, Amblyomma americanum, Amblyomma variegatum, Amblyomma maculatum, Amblyomma hebraeum, Amblyomma cajennense, Dermanyssus gallinae, Ornithonyssus bursa, Ornithonyssus sylviarum, and Varroa jacobsconi;
Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp.; specifically, Cheyletiella yasguri, Cheyletiella blakei, Demodex canis, Demodex bovis, Demodex ovis, Demodex caprae, Demodex equi, Demodex caballi, Demodex suis, Neotrombicula autumnalis, Neotrombicula desaleli, Neoschonegastia xerothermobia, Trombicula akamushi, Otodectes cynotis, Notoedres cati, Sarcoptis canis, Sarcoptes bovis, Sarcoptes ovis, Sarcoptes rupicaprae (=S. caprae), Sarcoptes equi, Sarcoptes suis, Psoroptes ovis, Psoroptes cuniculi, Psoroptes equi, Chorioptes bovis, Psoergates ovis, Pneumonyssoidic mange, Pneumonyssoides caninum, and Acarapis woodi;
nematodes, for example, Meloidogyne incognita, Bursaphelenchus xylophilus, Aphelenchoides besseyi, Heterodera glycines, Pratylenchus spp., etc; and
arthropods, helminths, and plasmodiums that invade animals. Preventing and controlling arthropods, helminths, and/or plasmodiums can decrease the mortality of domestic animals and improve the productivity (meat, milk, fur, skin, egg, and honey) and health of animals.

In a possible implementation, the animal parasite control agent is used for preventing and controlling one or more of Ctenocephalides felis and Dermacentor variabilis.

In a possible implementation, the animals include one or more of the following: agricultural animals, for example, cattle, sheep, goats, horses, pigs, donkeys, camels, water buffalo, rabbits, domestic chickens, turkeys, ducks, geese, farmed fishes, and bees, etc.; further include pets, which also called companion animals, for example, dogs, cats, cage birds, and ornamental fishes; and further include animals used for experiments, for example, hamsters, guinea pigs, rats, and mice, etc.

An embodiment of the present disclosure further provides an animal parasite control agent. The animal parasite control agent includes the phenylpyrazole compound as an active component, and further includes one or more excipients.

In a possible implementation, the animal parasite control agent is selected from the following dosage forms: tablet, capsule, portion, potable medicine, granule, ointment, pill, suppository, injection (muscular, subcutaneous, intravenous, and intraperitoneal, etc.), liniment, aerosol, and pressure-free spray (e.g., pump spray and atomized spray).

In a possible implementation, an amount of the active component contained in the animal parasite control agent is 1 to 80 wt %.

An embodiment of the present disclosure further provides an animal parasite control composition. The animal parasite control composition includes a mixture of the phenylpyrazole compound and other animal parasite control active compounds (e.g., an acaricide, an insecticide, a parasiticide, and an antimalarial agent, etc.). The mixture may be provided in the form of a bulk drug and may also be provided in the form of a commercially available formulation or in a use form prepared by the formulation thereof.

An embodiment of the present disclosure further provides a method for controlling animal parasites. The method includes the following step: administering an effective dose of a material to the animal parasites or a growth medium thereof to be controlled, where the material is selected from one or more of the following groups: the phenylpyrazole compound; the animal parasite control agent; and the animal parasite control composition. For example, intestinal administration is performed by using tablet, capsule, portion, potable medicine, granule, ointment, pill, or suppository; non-intestinal administration is performed based on a skin, for example, injection (muscular, subcutaneous, intravenous, or intraperitoneal, etc.), implantation, and nasal administration, including bathing or soaking, spraying, pouring, dripping, cleaning, and dusting; and administration is performed by using a model product containing the active compound, for example, collar, ear tag, label, leg brace, net, marker, etc. The active compound of the present disclosure has low toxicity and can be safely used in warm-blooded animals.

### Beneficial Effects

The phenylpyrazole compound of the present disclosure has an unexpected excellent insecticidal and acaricidal effect, also shows a suitable prevention and control effect on poisonous pests, and has no phytotoxicity to cultivated crop plants. In addition, the compound of the present disclosure can be used for preventing and controlling a variety of pests, for example, harmful piercing-sucking insects, chewing insects and other plant parasitic pests, stored grain pests, sanitary pests, etc., and can be used for disinfecting and killing the pests.

### DESCRIPTION OF THE EMBODIMENTS

In order to clarify the purpose, technical solutions and advantages of the embodiments of the present disclosure, the technical solutions of the embodiments of the present disclosure will be described clearly and completely below. Obviously, the embodiments described are a part of the embodiments of the present disclosure, rather than all of the embodiments. Based on the embodiments of the present disclosure, all other embodiments obtained by those of ordinary in the art without creative effort shall fall within the scope of protection of the present disclosure.

In addition, in order to better illustrate the present disclosure, numerous specific details are provided in the specific implementations hereinafter. Those skilled in the art shall understand that the present disclosure can also be implemented without certain specific details. In some embodiments, raw materials, elements, methods, means and the like that are well known to those skilled in the art are not described in detail, so as to conveniently highlight the purpose of the present disclosure.

Unless otherwise explicitly specified, in the entire description and claims, the term “comprise” or variants thereof, such as “include” or “comprising”, etc., will be understood as including the stated elements or components without excluding other elements or other components.

Unless otherwise indicated, all raw materials used are commercially available.

In the present disclosure, the terms used have the following meanings:

In definitions of compounds as shown in the general formulas given above, the terms used in the collection generally represent the following substituents:

Halogen: referring to fluorine, chlorine, bromine, or iodine.

Alkyl: linear or branched alkyl, for example, methyl, ethyl, n-propyl, isopropyl, or different butyl, pentyl, or hexyl isomers.

Haloalkyl: linear or branched alkyl on which hydrogen atoms may be partially or completely substituted with halogen, for example, monochloromethyl, dichloromethyl, trichloromethyl, monobromomethyl, dibromomethyl, tribromomethyl, monofluoromethyl, difluoromethyl, trifluoromethyl, 2,2,2-trifluoroethyl, heptafluoroisopropyl, or perfluoroethyl, etc.

Cycloalkyl: substituted or unsubstituted cyclic alkyl, for example, cyclopropyl, cyclobutyl, cyclopentyl, or cyclohexyl; substituents, for example, methyl, halogen, or cyanide, etc.

Haloalkoxy: alkoxy on which hydrogen atoms may be partially or completely substituted with halogen, for example, monofluoromethoxy, difluoromethoxy, trifluoromethoxy, or 2,2,2-trifluoroethoxy, etc.

Alkoxyalkyl: alkyl-O-alkyl-, for example, CH₃OCH₂—, CH₃CH₂OCH₂—, CH₃CH₂CH₂OCH₂—, (CH₃)₂CHOCH₂—, CH₃CH₂CH₂CH₂OCH₂—, (CH₃)₃COCH₂—, CH₃OCH₂CH₂—, CH₃CH₂OCH₂CH₂—, CH₃CH₂CH₂OCH₂CH₂—, or CH₃CH₂CH₂CH₂OCH₂CH₂—, etc.

Allyl: CH₂═CH—CH₂—.

Propargyl: CH≡C—CH₂—.

Animal parasite control agent: referring to active compound capable of effectively decreasing the incidence of various parasites in animals infected with parasites. Prevention and control means that the active compound can effectively kill the parasites and inhibit the growth or reproduction thereof.

Insecticide: substance having an insecticidal effect on pests.

Acaricide: agent used for preventing and controlling phytophagous acarids.

### Synthetic Embodiments

The compound of general formula I of the present disclosure can be prepared separately by using different raw material compounds according to the synthetic routes recorded above, which is further described specifically as follows.

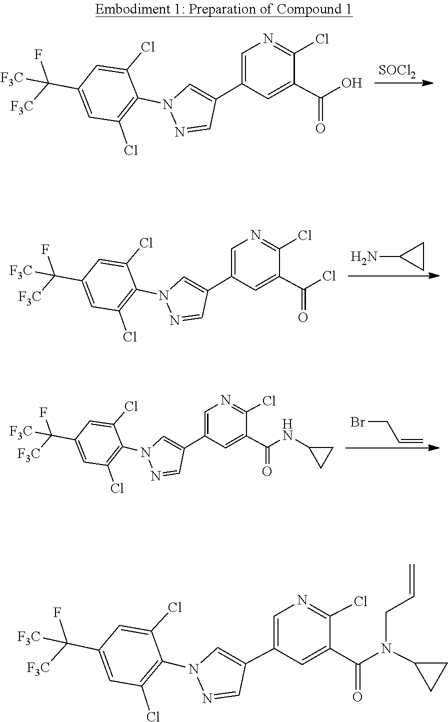

### Step 1: Synthesis of Intermediate 2-chloro-5-{1-[2,6-dichloro-4-(perfluoropropan-2-yl)phenyl]-1H-pyrazol-4-yl}nicotinoyl chloride

2-chloro-5-{1-[2,6-dichloro-4-(perfluoropropan-2-yl)phenyl]-1H-pyrazol-4-yl}nicotinic acid (20.00 g, 37.27 mmol), thionyl chloride (13.30 g, 111.81 mmol) and 100 mL of toluene were added into a 250 mL reaction flask, heated to 110° C. and reacted for 3 h. The reaction solution was concentrated under reduced pressure to obtain 20.53 g of an oily compound, that is, the intermediate 2-chloro-5-t{1-[2,6-dichloro-4-(perfluoropropan-2-yl)phenyl]-1H-pyrazol-4-yl}nicotinoyl chloride.

### Step 2: Synthesis of Intermediate 2-chloro-N-cyclopropyl-5-{1-[2,6-dichloro-4-(perfluoropropan-2-yl)phenyl]-1H-pyrazol-4-yl}nicotinamide

Cyclopropylamine (1.13 g, 19.79 mmol), triethylamine (4.01 g, 39.62 mmol) and 50 mL of dichloromethane were added into a 100 mL reaction flask, and 2-chloro-5-{1-[2,6-dichloro-4-(perfluoropropan-2-yl)phenyl]-1H-pyrazol-4-yl}nicotinoyl chloride (10.00 g, 18.02 mmol) was added dropwise under stirring in an ice bath. After the dropping was completed, the reaction solution was heated to room temperature and continued to react for 5 h. After the reaction monitored by TLC was completed, the reaction solution was concentrated to dryness under reduced pressure, and 150 mL of ethyl acetate and 150 mL of water were added for extraction. Organic phase was sequentially washed by a saturated sodium bicarbonate solution and saturated salt water, dried with anhydrous magnesium sulfate, and concentrated under reduced pressure. Residue was purified by column chromatography to obtain 8.21 g of a white solid, that is, the intermediate 2-chloro-N-cyclopropyl-5-t{1-[2,6-dichloro-4-(perfluoropropan-2-yl)phenyl]-1H-pyrazol-4-yl}nicotinamide.

### Step 3: Synthesis of Compound 1

60% NaH (0.03 g, 0.78 mmol) and 10 mL of THF were added into a 25 mL reaction flask and cooled to 0-5° C. in an ice bath. 2-chloro-N-cyclopropyl-5-{1-[2,6-dichloro-4-(perfluoropropan-2-yl)phenyl]-1H-pyrazol-4-yl}nicotinamide (0.30 g, 0.52 mmol) was added under stirring, and after maintaining at 0-5° C. and stirring for 30 min, allyl bromide (0.12 g, 0.99 mmol) was added. The reaction solution was heated to room temperature and continuously stirred to react for 3 h. After the reaction monitored by TLC was completed, 20 mL of water was added to quench the reaction. 50 mL of ethyl acetate and 50 mL of water were added to the reaction solution for extraction. Organic phase was sequentially washed by saturated salt water, dried with anhydrous magnesium sulfate, filtered, and concentrated under reduced pressure. Residue was purified by column chromatography to obtain 0.28 g of a white solid, that is, target Compound 1. Nuclear magnetic resonance and mass spectrometry data of Compound 1 are as follows:

¹H NMR (600 MHz, DMSO-d₆) δ 8.85 (s, 1H), 8.85-8.80 (m, 1H), 8.56 (s, 1H), 8.30 (d, 1H), 8.11 (s, 2H), 6.00-5.88 (m, 1H), 5.36-5.28 (m, 1H), 5.25-5.18 (m, 1H), 4.13 (d, 2H), 2.79-2.70 (m, 1H), 0.65-0.60 (m, 2H), 0.54-0.49 (m, 2H). ESI-MS(m/z): 615.13 [M+H]⁺.

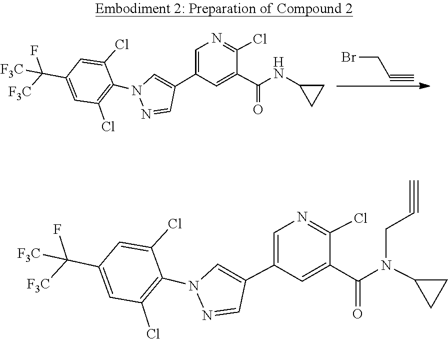

Referring to the synthesis method in Step 3 of Embodiment 1, target Compound 2 (white solid) was prepared by using 2-chloro-N-cyclopropyl-5-{1-[2,6-dichloro-4-(perfluoropropan-2-yl)phenyl]-1H-pyrazol-4-yl}nicotinamide and propargyl bromide as reaction raw materials. Nuclear magnetic resonance and mass spectrometry data of Compound 2 are as follows:

¹H NMR (400 MHz, Chloroform-d) δ 8.64 (d, 1H), 8.13 (s, 1H), 7.92 (s, 1H), 7.78 (d, 1H), 7.75 (s, 2H), 4.39 (s, 2H), 3.01-2.92 (m, 1H), 2.29 (t, 1H), 0.93-0.77 (m, 2H), 0.74-0.59 (m, 2H). ESI-MS(m/z): 613.13 [M+H]⁺.

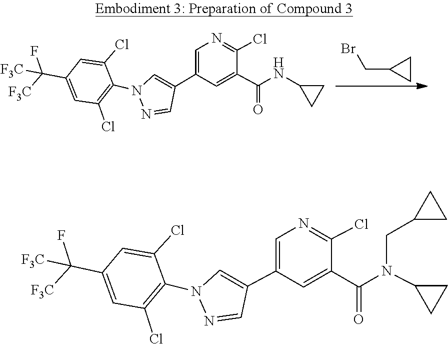 [SMILES: O=C(NC1CC1)c1cc(-c2cnn(-c3c(Cl)cc(C(F)(C(F)(F)F)C(F)(F)F)cc3Cl)c2)cnc1Cl ; BrCC1CC1 ; O=C(c1cc(-c2cnn(-c3c(Cl)cc(C(F)(C(F)(F)F)C(F)(F)F)cc3Cl)c2)cnc1Cl)N(CC1CC1)C1CC1]

Referring to the synthesis method in Step 3 of Embodiment 1, target Compound 3 (oily compound) was prepared by using 2-chloro-N-cyclopropyl-5-t{1-[2,6-dichloro-4-(perfluoropropan-2-yl)phenyl]-1H-pyrazol-4-yl}nicotinamide and cyclopropyl bromide as reaction raw materials. Nuclear magnetic resonance and mass spectrometry data of Compound 3 are as follows:

¹H NMR (600 MHz, Chloroform-d) δ 8.62 (d, 1H), 8.13 (s, 1H), 7.92 (s, 1H), 7.77 (d, 1H), 7.75 (s, 2H), 3.91-3.74 (m, 1H), 3.21-3.04 (m, 1H), 3.03-2.94 (m, 2H), 0.72-0.54 (m, 4H), 0.47-0.33 (m, 4H). ESI-MS(m/z): 629.12 [M+H]⁺.

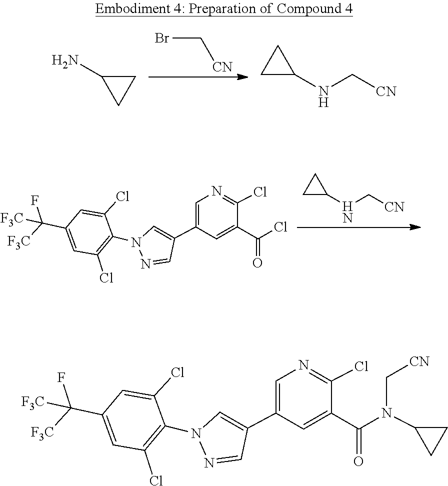

### Step 1: Synthesis of Intermediate 2-(cyclopropyl)aminoacetonitrile

Cyclopropylamine (1.00 g, 17.52 mmol), bromoacetonitrile (4.20 g, 35.03 mmol), N,N-diisopropylethylamine (6.79 g, 52.55 mmol) and 10 mL of toluene were added into a 100 mL reaction flask, and the reaction solution was heated to reflux and continued to react for 3 h. After the reaction monitored by TLC was completed, the reaction solution was concentrated to dryness under reduced pressure, and 50 mL of ethyl acetate and 50 mL of water were added for extraction. Organic phase was washed by saturated salt water, dried with anhydrous magnesium sulfate, and concentrated under reduced pressure to obtain 0.99 g of an oily compound, that is, intermediate 2-(cyclopropyl)aminoacetonitrile.

### Step 2: Synthesis of Compound 4

2-(cyclopropyl)aminoacetonitrile (0.99 g, 10.31 mmol), triethylamine (2.09 g, 20.62 mmol) and 50 mL of dichloromethane were added into a 100 mL reaction flask, and 2-chloro-5-{1-[2,6-dichloro-4-(perfluoropropan-2-yl)phenyl]-1H-pyrazol-4-yl}nicotinoyl chloride (5.70 g, 10.31 mmol) was added dropwise under stirring in an ice bath. After the dropping was completed, the reaction solution was heated to room temperature and continued to react for 4 h. After the reaction monitored by TLC was completed, the reaction solution was concentrated to dryness under reduced pressure, and 150 mL of ethyl acetate and 150 mL of water were added for extraction. Organic phase was sequentially washed by a saturated sodium bicarbonate solution and saturated salt water, dried with anhydrous magnesium sulfate, and concentrated under reduced pressure. Residue was purified by column chromatography to obtain 4.87 g of a white solid, that is, target Compound 4. Nuclear magnetic resonance and mass spectrometry data of Compound 4 are as follows:

¹H NMR (600 MHz, Chloroform-d) δ 8.68 (d, 1H), 8.15 (s, 1H), 7.95 (s, 1H), 7.80 (d, 1H), 7.76 (s, 2H), 4.51 (s, 2H), 3.06-2.96 (m, 1H), 0.84-0.63 (m, 4H). ESI-MS(m/z): 614.03 [M+H]⁺.

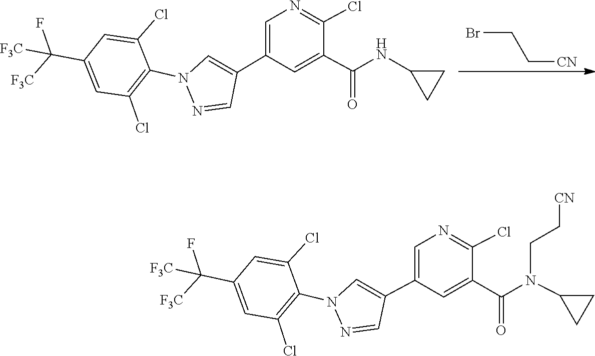 [SMILES: O=C(NC1CC1)c1cc(-c2cnn(-c3c(Cl)cc(C(F)(C(F)(F)F)C(F)(F)F)cc3Cl)c2)cnc1Cl ; N#CCCBr ; N#CCCN(C(=O)c1cc(-c2cnn(-c3c(Cl)cc(C(F)(C(F)(F)F)C(F)(F)F)cc3Cl)c2)cnc1Cl)C1CC1]

Referring to the synthesis method in Step 3 of Embodiment 1, target Compound 5 (white solid) was prepared by using 2-chloro-N-cyclopropyl-5-{1-[2,6-dichloro-4-(perfluoropropan-2-yl)phenyl]-1H-pyrazol-4-yl}nicotinamide and 3-bromopropionitrile as reaction raw materials. Nuclear magnetic resonance and mass spectrometry data of Compound 5 are as follows:

¹H NMR (600 MHz, Chloroform-d) δ 8.67 (d, 1H), 8.15 (s, 1H), 7.95 (s, 1H), 7.83 (d, 1H), 7.76 (s, 2H), 3.86 (s, 2H), 3.06-2.98 (m, 1H), 2.89 (s, 2H), 0.92-0.38 (m, 4H). ESI-MS(m/z): 628.14 [M+H]⁺.

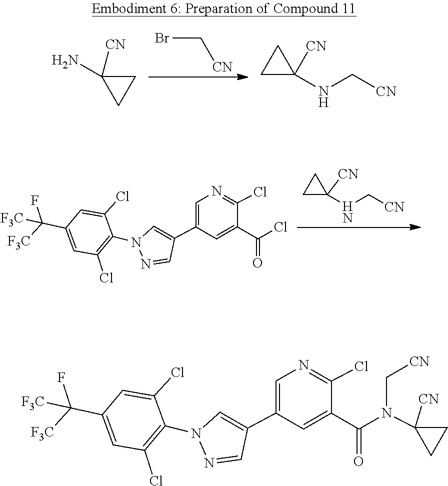

### Step 1: Synthesis of Intermediate 1-((cyanomethyl)amino)cyclopropan-1-nitrile

Referring to the synthesis method in Step 1 of Embodiment 4, the intermediate 1-((cyanomethyl)amino)cyclopropan-1-nitrile was prepared by using 1-aminocyclopropan-1-nitrile and bromoacetonitrile as raw materials.

### Step 2: Synthesis of Compound 11

Referring to the synthesis method in Step 2 of Embodiment 4, target Compound 11 (white solid) was prepared by using 2-chloro-5-{1-[2,6-dichloro-4-(perfluoropropan-2-yl)phenyl]-1H-pyrazol-4-yl}nicotinoyl chloride and 1-((cyanomethyl)amino)cyclopropan-1-nitrile as raw materials. Nuclear magnetic resonance and mass spectrometry data of Compound 11 are as follows:

¹H NMR (600 MHz, Chloroform-d) δ 8.77 (d, 1H), 8.21 (s, 1H), 8.10 (s, 1H), 8.00 (s, 1H), 7.74 (s, 2H), 4.57 (s, 2H), 1.66 (s, 2H), 1.43 (s, 2H). ESI-MS(m/z): 639.07 [M+H]⁺.

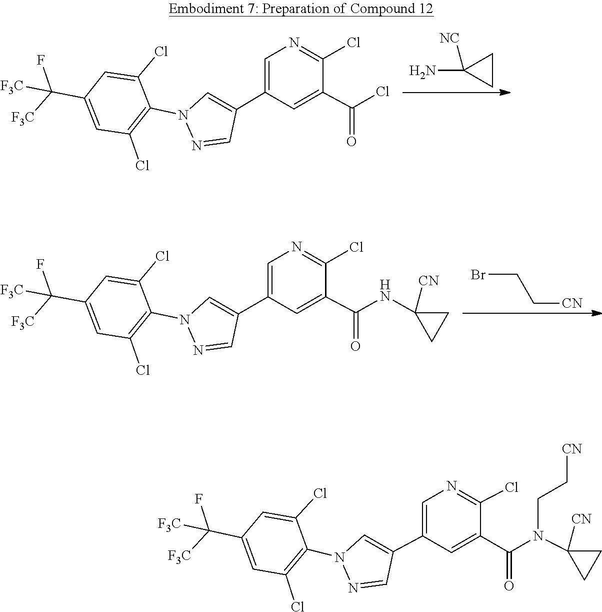 [SMILES: O=C(Cl)c1cc(-c2cnn(-c3c(Cl)cc(C(F)(C(F)(F)F)C(F)(F)F)cc3Cl)c2)cnc1Cl ; N#CC1(N)CC1 ; N#CC1(NC(=O)c2cc(-c3cnn(-c4c(Cl)cc(C(F)(C(F)(F)F)C(F)(F)F)cc4Cl)c3)cnc2Cl)CC1 ; N#CCCBr ; N#CCCN(C(=O)c1cc(-c2cnn(-c3c(Cl)cc(C(F)(C(F)(F)F)C(F)(F)F)cc3Cl)c2)cnc1Cl)C1(C#N)CC1]

### Step 1: Synthesis of Intermediate 2-chloro-N-(1-cyanocyclopropyl)-5-{1-[2,6-dichloro-4-(perfluoropropyl)phenyl]-1H-pyrazol-4-yl}nicotinamide

cyanocyclopropylamine (1 g, 12.18 mmol), triethylamine (2.46 g, 24.36 mmol) and 50 mL of dichloromethane were added into a 100 mL reaction flask, and 2-chloro-5-{1-[2,6-dichloro-4-(perfluoropropan-2-yl)phenyl]-1H-pyrazol-4-yl}nicotinoyl chloride (6.74 g, 12.18 mmol) was added dropwise under stirring in an ice bath. After the dropping was completed, the reaction solution was heated to room temperature and continued to react for 5 h. After the reaction monitored by TLC was completed, the reaction solution was concentrated to dryness under reduced pressure, and 150 mL of ethyl acetate and 150 mL of water were added for extraction. Organic phase was sequentially washed by a saturated sodium bicarbonate solution and saturated salt water, dried with anhydrous magnesium sulfate, and concentrated under reduced pressure. Residue was purified by column chromatography to obtain 5.14 g of a white solid, that is, intermediate 2-chloro-N-(1-cyanocyclopropyl)-5-{1-[2,6-dichloro-4-(perfluoropropyl)phenyl]-1H-pyrazol-4-yl}nicotinamide.

### Step 2: Synthesis of Compound 12

60% NaH (0.03 g, 0.78 mmol) and 10 mL of THF were added into a 25 mL reaction flask and cooled to 0-5° C. in an ice bath. 2-chloro-N-(1-cyanocyclopropyl)-5-{1-[2,6-dichloro-4-(perfluoropropyl)phenyl]-1H-pyrazol-4-yl}nicotinamide (0.31 g, 0.52 mmol) was added under stirring, and after maintaining at 0-5° C. and stirring for 30 min, 3-bromo-propionitrile (0.14 g, 1.04 mmol) was added. The reaction solution was heated to room temperature and continuously stirred to react for 4 h. After the reaction monitored by TLC was completed, 20 mL of water was added to quench the reaction. 50 mL of ethyl acetate and 50 mL of water were added to the reaction solution for extraction. Organic phase was sequentially washed by saturated salt water, dried with anhydrous magnesium sulfate, filtered, and concentrated under reduced pressure. Residue was purified by column chromatography to obtain 0.28 g of a white solid, that is, target Compound 12. Nuclear magnetic resonance and mass spectrometry data of Compound 12 are as follows:

¹H NMR (600 MHz, DMSO-d₆) δ 8.92 (d, 1H), 8.80 (d, 1H), 8.54-8.40 (m, 1H), 8.29 (s, 1H), 8.10 (s, 2H), 3.58 (d, 2H), 3.09-2.79 (m, 2H), 1.78 (d, 2H), 1.60-1.33 (m, 2H). ESI-MS(m/z): 65316 [M+H]⁺.

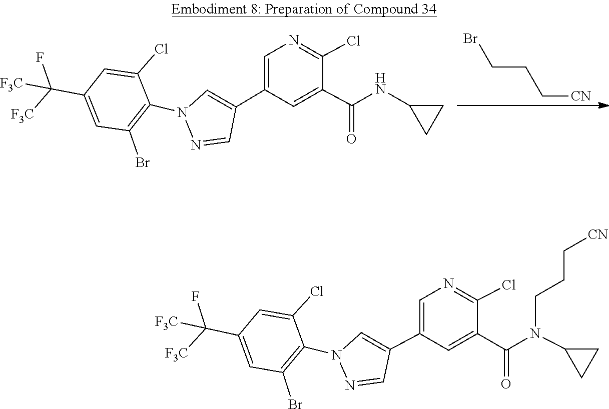 [SMILES: O=C(NC1CC1)c1cc(-c2cnn(-c3c(Cl)cc(C(F)(C(F)(F)F)C(F)(F)F)cc3Br)c2)cnc1Cl ; N#CCCCBr ; N#CCCCN(C(=O)c1cc(-c2cnn(-c3c(Cl)cc(C(F)(C(F)(F)F)C(F)(F)F)cc3Br)c2)cnc1Cl)C1CC1]

Referring to the synthesis method in Step 3 of Embodiment 1, target compound 34 (brown solid) was prepared by using 5-{1-[2-bromo-6-chloro-4-(perfluoropropyl)phenyl]-1H-pyrazol-4-yl}-2-chloro-N-cyclopropyl nicotinamide and 4-bromo-butyronitrile as reaction raw materials. Nuclear magnetic resonance and mass spectrometry data of Compound 34 are as follows:

¹H NMR (600 MHz, Chloroform-d) δ 8.65 (d, 1H), 8.14 (s, 1H), 7.95-7.88 (m, 3H), 7.79 (d, 1H), 3.72 (d, 2H), 2.92-2.84 (m, 1H), 2.62-2.50 (m, 2H), 2.20-2.09 (m, 2H), 0.96-0.39 (m, 4H). ESI-MS(m/z): 686.01 [M+H]⁺.

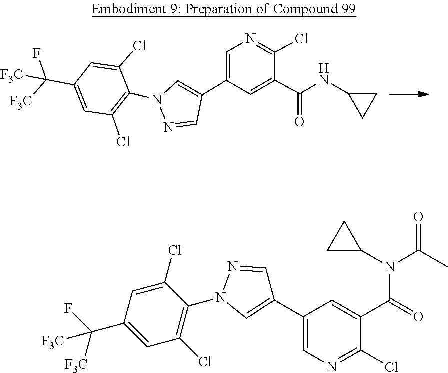 [SMILES: O=C(NC1CC1)c1cc(-c2cnn(-c3c(Cl)cc(C(F)(C(F)(F)F)C(F)(F)F)cc3Cl)c2)cnc1Cl ; CC(=O)N(C(=O)c1cc(-c2cnn(-c3c(Cl)cc(C(F)(C(F)(F)F)C(F)(F)F)cc3Cl)c2)cnc1Cl)C1CC1]

60% NaH (0.03 g, 0.78 mmol) and 10 mL of THF were added into a 25 mL reaction flask and cooled to 0-5° C. in an ice bath. 2-chloro-N-cyclopropyl-5-{1-[2,6-dichloro-4-(perfluoropropan-2-yl)phenyl]-1H-pyrazol-4-yl}nicotinamide (0.30 g, 0.52 mmol) was added under stirring, and after maintaining at 0-5° C. and stirring for 30 min, acetyl chloride (0.09 g, 1.17 mmol) was added. The reaction solution was heated to room temperature and continuously stirred to react for 2 h. After the reaction monitored by TLC was completed, 20 mL of water was added to quench the reaction. 50 mL of ethyl acetate and 50 mL of water were added to the reaction solution for extraction. Organic phase was sequentially washed by saturated salt water, dried with anhydrous magnesium sulfate, filtered, and concentrated under reduced pressure. Residue was purified by column chromatography to obtain 0.27 g of a white solid, that is, target Compound 99. Nuclear magnetic resonance and mass spectrometry data of Compound 99 are as follows:

¹H NMR (600 MHz, Chloroform-d) δ 8.63 (d, 1H), 8.14 (s, 1H), 7.93 (s, 1H), 7.83 (d, 1H), 7.75 (s, 2H), 2.87-2.82 (m, 1H), 2.51 (s, 3H), 1.14-1.07 (m, 2H), 0.87-0.80 (m, 2H). ESI-MS(m/z): 617.04 [M+H]⁺.

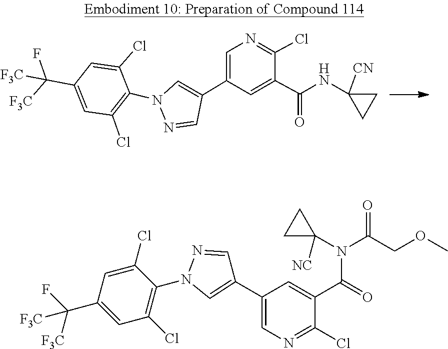 [SMILES: N#CC1(NC(=O)c2cc(-c3cnn(-c4c(Cl)cc(C(F)(C(F)(F)F)C(F)(F)F)cc4Cl)c3)cnc2Cl)CC1 ; COCC(=O)N(C(=O)c1cc(-c2cnn(-c3c(Cl)cc(C(F)(C(F)(F)F)C(F)(F)F)cc3Cl)c2)cnc1Cl)C1(C#N)CC1]

Referring to the synthesis method in Embodiment 9, target Compound 114 (white solid) was prepared by using 2-chloro-N-(1-cyanocyclopropyl)-5-{1-[2,6-dichloro-4-(perfluoropropyl)phenyl]-1H-pyrazol-4-yl}nicotinamide and 2-methoxyacetyl chloride as reaction raw materials. Nuclear magnetic resonance and mass spectrometry data of Compound 114 are as follows:

¹H NMR (600 MHz, Chloroform-d) δ 8.75 (d, 1H), 8.20 (d, 1H), 8.08 (d, 1H), 8.01 (d, 1H), 7.74 (s, 2H), 4.53 (s, 2H), 3.50 (s, 3H), 1.74 (s, 2H), 1.52-1.48 (m, 2H). ESI-MS(m/z): 672.10 [M+H]⁺.

Chemical structural formulas, physical and chemical properties, and nuclear magnetic resonance and mass spectrometry data of some compounds of the present disclosure are as follows:

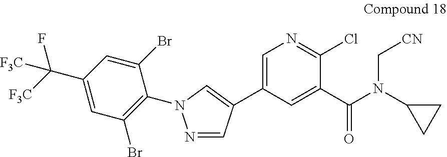 [SMILES: N#CCN(C(=O)c1cc(-c2cnn(-c3c(Br)cc(C(F)(C(F)(F)F)C(F)(F)F)cc3Br)c2)cnc1Cl)C1CC1]

White solid. ¹H NMR (600 MHz, Chloroform-d) δ 8.69 (d, 1H), 8.14 (s, 1H), 7.95 (s, 2H), 7.93 (s, 1H), 7.81 (d, 1H), 4.51 (s, 2H), 3.07-2.99 (m, 1H), 0.93-0.53 (m, 4H). ESI-MS(m/z): 702.03 [M+H]⁺.

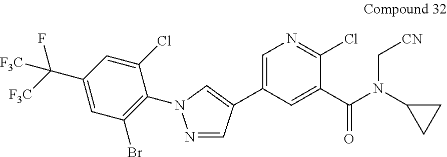 [SMILES: N#CCN(C(=O)c1cc(-c2cnn(-c3c(Cl)cc(C(F)(C(F)(F)F)C(F)(F)F)cc3Br)c2)cnc1Cl)C1CC1]

Brown solid. ¹H NMR (600 MHz, Chloroform-d) δ 8.69 (d, 1H), 8.15 (s, 1H), 7.94 (s, 1H), 7.91 (s, 1H), 7.80 (d, 2H), 4.51 (s, 2H), 3.05-2.99 (m, 1H), 0.93-0.53 (m, 4H). ESI-MS(m/z): 657.93 [M+H]⁺.

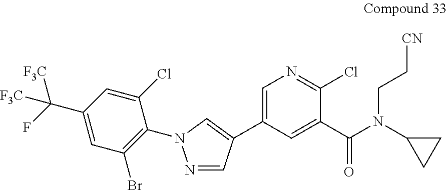 [SMILES: N#CCCN(C(=O)c1cc(-c2cnn(-c3c(Cl)cc(C(F)(C(F)(F)F)C(F)(F)F)cc3Br)c2)cnc1Cl)C1CC1]

White solid. ¹H NMR (600 MHz, Chloroform-d) δ 8.66 (d, 1H), 8.14 (s, 1H), 7.93 (s, 1H), 7.91 (d, 1H), 7.82 (d, 1H), 7.79 (d, 1H), 3.88 (s, 2H), 3.06-2.99 (m, 1H), 2.89 (s, 2H), 0.91-0.42 (m, 4H). ESI-MS(m/z): 672.14 [M+H]⁺.

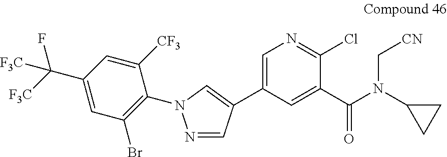 [SMILES: N#CCN(C(=O)c1cc(-c2cnn(-c3c(Br)cc(C(F)(C(F)(F)F)C(F)(F)F)cc3C(F)(F)F)c2)cnc1Cl)C1CC1]

Yellow solid. ¹H NMR (600 MHz, DMSO-d₆) δ 8.88 (d, 1H), 8.85 (s, 1H), 8.57 (d, 1H), 8.55 (s, 1H), 8.39 (d, 1H), 8.12 (d, 1H), 4.64 (s, 2H), 2.94-2.87 (m, 1H), 0.77-0.67 (m, 2H), 0.66-0.52 (m, 2H). ESI-MS(m/z): 692.00 [M+H]⁺.

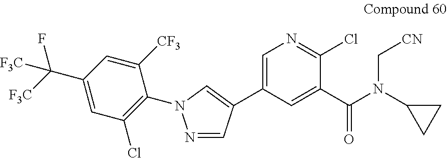 [SMILES: N#CCN(C(=O)c1cc(-c2cnn(-c3c(Cl)cc(C(F)(C(F)(F)F)C(F)(F)F)cc3C(F)(F)F)c2)cnc1Cl)C1CC1]

White solid. ¹H NMR (600 MHz, Chloroform-d) δ 8.68 (d, 1H), 8.14 (s, 1H), 8.05 (d, 1H), 7.99 (d, 1H), 7.95 (s, 1H), 7.80 (d, 1H), 4.51 (s, 2H), 3.06-2.99 (m, 1H), 0.93-0.53 (m, 4H). ESI-MS(m/z): 648.29 [M+H]⁺.

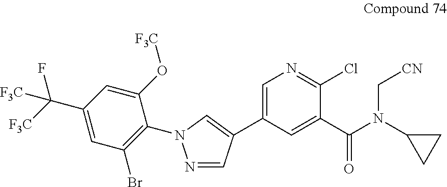 [SMILES: N#CCN(C(=O)c1cc(-c2cnn(-c3c(Br)cc(C(F)(C(F)(F)F)C(F)(F)F)cc3OC(F)(F)F)c2)cnc1Cl)C1CC1]

White solid. ¹H NMR (600 MHz, Chloroform-d) δ 8.68 (d, 1H), 8.14 (s, 1H), 7.96 (d, 1H), 7.94 (s, 1H), 7.80 (d, 1H), 7.67 (s, 1H), 4.51 (s, 2H), 3.06-2.99 (m, 1H), 0.91-0.68 (m, 4H). ESI-MS(m/z): 708.08 [M+H]⁺.

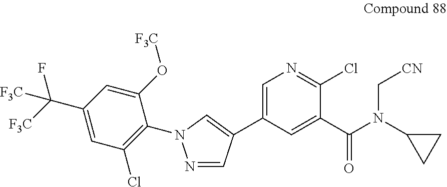 [SMILES: N#CCN(C(=O)c1cc(-c2cnn(-c3c(Cl)cc(C(F)(C(F)(F)F)C(F)(F)F)cc3OC(F)(F)F)c2)cnc1Cl)C1CC1]

White solid. ¹H NMR (600 MHz, Chloroform-d) δ 8.68 (d, 1H), 8.14 (s, 1H), 7.95 (d, 1H), 7.81 (d, 1H), 7.79 (d, 1H), 7.63 (s, 1H), 4.51 (s, 2H), 3.06-2.99 (m, 1H), 0.91-0.68 (m, 4H). ESI-MS(m/z): 664.21 [M+H]⁺.

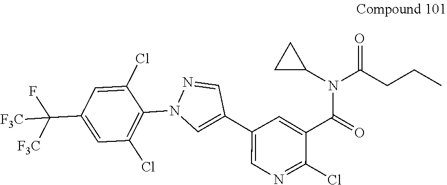 [SMILES: CCCC(=O)N(C(=O)c1cc(-c2cnn(-c3c(Cl)cc(C(F)(C(F)(F)F)C(F)(F)F)cc3Cl)c2)cnc1Cl)C1CC1]

White solid. ¹H NMR (600 MHz, Chloroform-d) δ 8.62 (d, 1H), 8.13 (s, 1H), 7.92 (s, 1H), 7.82 (d, 1H), 7.75 (s, 2H), 2.87-2.80 (m, 3H), 1.75-1.64 (m, 2H), 1.16-1.09 (m, 2H), 0.98 (t, 3H), 0.89-0.80 (m, 2H). ESI-MS(m/z): 645.11 [M+H]⁺.

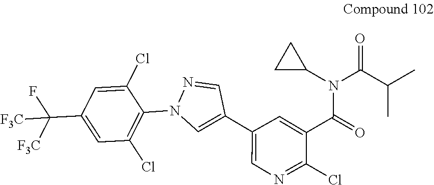 [SMILES: CC(C)C(=O)N(C(=O)c1cc(-c2cnn(-c3c(Cl)cc(C(F)(C(F)(F)F)C(F)(F)F)cc3Cl)c2)cnc1Cl)C1CC1]

White solid. ¹H NMR (600 MHz, Chloroform-d) δ 8.63 (d, 1H), 8.13 (s, 1H), 7.92 (s, 1H), 7.80 (d, 1H), 7.75 (s, 2H), 3.59 (hept, 1H), 2.90-2.83 (m, 1H), 1.21 (d, 6H), 1.12-1.07 (m, 2H), 0.83-0.78 (m, 2H). ESI-MS(m/z): 645.13 [M+H]⁺.

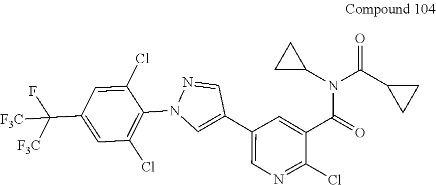 [SMILES: O=C(c1cc(-c2cnn(-c3c(Cl)cc(C(F)(C(F)(F)F)C(F)(F)F)cc3Cl)c2)cnc1Cl)N(C(=O)C1CC1)C1CC1]

White solid. ¹H NMR (600 MHz, Chloroform-d) δ 8.59 (d, 1H), 8.11 (s, 1H), 7.90 (s, 1H), 7.79 (d, 1H), 7.74 (s, 2H), 3.03-2.97 (m, 1H), 2.52-2.44 (m, 1H), 1.24-1.19 (m, 2H), 1.12-1.08 (m, 2H), 1.07-1.02 (m, 2H), 0.99-0.95 (m, 2H). ESI-MS(m/z): 643.07 [M+H]⁺.

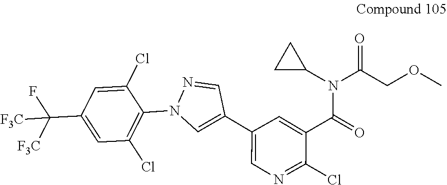 [SMILES: COCC(=O)N(C(=O)c1cc(-c2cnn(-c3c(Cl)cc(C(F)(C(F)(F)F)C(F)(F)F)cc3Cl)c2)cnc1Cl)C1CC1]

White solid. ¹H NMR (600 MHz, Chloroform-d) δ 8.68 (d, 1H), 8.15 (s, 1H), 7.95 (s, 1H), 7.86 (d, 1H), 7.76 (s, 2H), 4.57 (s, 2H), 3.50 (s, 3H), 2.86-2.78 (m, 1H), 1.03-0.96 (m, 2H), 0.76-0.70 (m, 2H). ESI-MS(m/z): 647.10 [M+H]⁺.

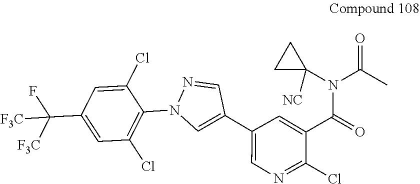 [SMILES: CC(=O)N(C(=O)c1cc(-c2cnn(-c3c(Cl)cc(C(F)(C(F)(F)F)C(F)(F)F)cc3Cl)c2)cnc1Cl)C1(C#N)CC1]

White solid. ¹H NMR (600 MHz, Chloroform-d) δ 8.69 (d, 1H), 8.17 (s, 1H), 7.99-7.97 (m, 2H), 7.74 (s, 2H), 2.58 (s, 3H), 1.87 (s, 2H), 1.43 (s, 2H). ESI-MS(m/z): 642.10 [M+H]⁺.

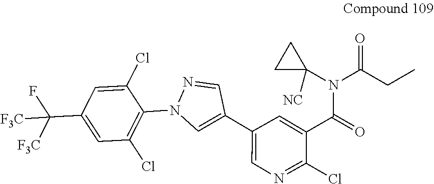 [SMILES: CCC(=O)N(C(=O)c1cc(-c2cnn(-c3c(Cl)cc(C(F)(C(F)(F)F)C(F)(F)F)cc3Cl)c2)cnc1Cl)C1(C#N)CC1]

White solid. ¹H NMR (600 MHz, DMSO-d₆) δ 8.86 (d, 1H), 8.80 (s, 1H), 8.50 (s, 1H), 8.33 (d, 1H), 8.11 (s, 2H), 2.88 (s, 2H), 2.01-1.89 (m, 2H), 1.26-1.10 (t, 2H), 1.04 (t, 3H). ESI-MS(m/z): 656.03 [M+H]⁺.

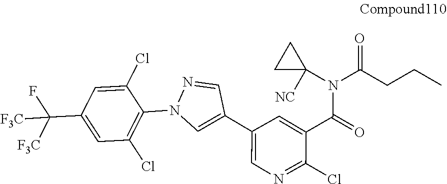 [SMILES: CCCC(=O)N(C(=O)c1cc(-c2cnn(-c3c(Cl)cc(C(F)(C(F)(F)F)C(F)(F)F)cc3Cl)c2)cnc1Cl)C1(C#N)CC1]

White solid. ¹H NMR (600 MHz, DMSO-d₆) δ 8.86 (d, 1H), 8.80 (s, 1H), 8.50 (s, 1H), 8.33 (d, 1H), 8.11 (s, 2H), 2.84 (t, 2H), 1.97 (s, 2H), 1.63-1.52 (m, 2H), 1.40 (s, 2H), 0.91 (t, 3H). ESI-MS(m/z): 670.11 [M+H]⁺.

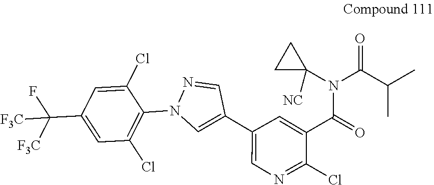 [SMILES: CC(C)C(=O)N(C(=O)c1cc(-c2cnn(-c3c(Cl)cc(C(F)(C(F)(F)F)C(F)(F)F)cc3Cl)c2)cnc1Cl)C1(C#N)CC1]

White solid. ¹H NMR (600 MHz, Chloroform-d) δ 8.70 (d, 1H), 8.17 (s, 1H), 7.99-7.95 (m, 2H), 7.74 (s, 2H), 3.50 (hept, 1H), 1.83 (s, 2H), 1.53-1.48 (m, 2H), 1.27 (d, 6H). ESI-MS(m/z): 670.19 [M+H]⁺.

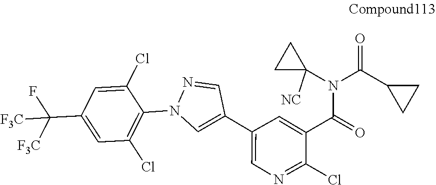 [SMILES: N#CC1(N(C(=O)c2cc(-c3cnn(-c4c(Cl)cc(C(F)(C(F)(F)F)C(F)(F)F)cc4Cl)c3)cnc2Cl)C(=O)C2CC2)CC1]

White solid. ¹H NMR (600 MHz, Chloroform-d) δ 8.64 (d, 1H), 8.14 (s, 1H), 7.93 (s, 1H), 7.88 (d, 1H), 7.74 (s, 2H), 2.39-2.31 (m, 1H), 2.03-1.95 (m, 2H), 1.71-1.63 (m, 2H), 1.24-1.12 (m, 4H). ESI-MS(m/z): 668.20 [M+H]⁺.

Other compounds as shown in the general formula I of the present disclosure can be prepared with reference to the above embodiments.

### Biological Activity Determination

### Embodiment 11: Biological Activity Determination of Mythimna separata and Plutella xylostella

Insecticidal activity determination experiments were carried out on several insects by using compounds of the present disclosure. The determination method is as follows:

The compounds to be tested were dissolved in a mixed solvent of acetone/methanol (1:1) and then diluted to desired concentrations with water containing 0.1% (wt) Tween 80.

With Mythimna separata and Plutella xylostella as targets, the activity was determined by Airbrush spray method.

(1) Activity Determination of Killing Mythimna separata

Determination method: Corn leaves were cut into leaf segments with the length of 2 cm, the pressure of Airbrush spray treatment is 10 psi (about 0.7 kg/cm²), and the compounds to be tested were sprayed onto the front and back surfaces of each leaf segment at a liquid amount of 0.5 mL. After drying in the shade, 10 3rd instar larvae were inoculated to each treatment, and each treatment was repeated for 3 times. After the treatment, the larvae were placed in an observation room for culture at 25° C. with relative humidity of 60-70%. On the 3^{rd} day after administration, the number of surviving larvae was investigated, and the mortality was calculated.

Some test results of Mythimna separata are as follows:

At the dose of 1.25 mg/L, the mortality of compounds 1, 2, 3, 4, 5, 11, 12, 18, 32, 33, 34, 46, 60, 74, 88, 99, 101, 102, 104, 105, 108, 109, 110, 111, 113 and 114 against Mythimna separata is 90% or above on 3^{rd} day after administration.

(2) Activity Determination of Killing Plutella xylostella

Determination method: Cabbage leaves were punched into leaf discs with the diameter of 2 cm by a puncher, the pressure of Airbrush spray treatment is 10 psi (about 0.7 kg/cm²), and the compounds to be tested were sprayed onto the front and back surfaces of each leaf disc at a liquid amount of 0.5 mL. After drying in the shade, 10 3rd instar larvae were inoculated to each treatment, and each treatment was repeated for 3 times. After the treatment, the larvae were placed in an observation room for culture at 25° C. with relative humidity of 60-70%. On 3^{rd} day after administration, the number of surviving larvae was investigated, and the mortality was calculated.

Some test results of Plutella xylostella are as follows:

At the dose of 1.25 mg/L, the mortality of compounds 1, 2, 3, 4, 5, 11, 12, 18, 32, 33, 34, 46, 60, 74, 88, 99, 101, 102, 104, 105, 108, 109, 110, 111, 113 and 114 against Plutella xylostella is 90% or above on 3^{rd} day after administration.

Some compounds of the present disclosure and control compounds were selected to carry out parallel comparative experiments of the insecticidal activity against Plutella xylostella (on 3^{rd} day after administration), and the determination method was the same as above. Results are shown in Table 5:

TABLE 5
Parallel comparative experiments of the insecticidal activity of some compounds of the
present disclosure and control compounds against Plutella xylostella

| Compound | | Mortality (%, on 3^{rd} day after administration) | | |
|---|---|---|---|---|
| Number | Structural Formula | 1.25 mg/L | 0.625 mg/L | 0.3125 mg/L |
| 1 | 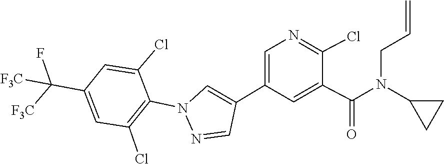 [SMILES: C=CCN(C(=O)c1cc(-c2cnn(-c3c(Cl)cc(C(F)(C(F)(F)F)C(F)(F)F)cc3Cl)c2)cnc1Cl)C1CC1] | 100 | 100 | 93.3 |
| 2 | 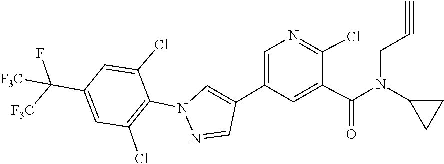 [SMILES: C#CCN(C(=O)c1cc(-c2cnn(-c3c(Cl)cc(C(F)(C(F)(F)F)C(F)(F)F)cc3Cl)c2)cnc1Cl)C1CC1] | 100 | 100 | 90 |
| 3 | 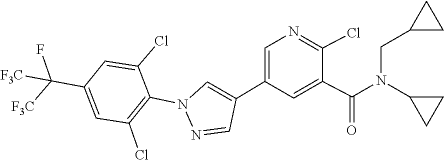 [SMILES: O=C(c1cc(-c2cnn(-c3c(Cl)cc(C(F)(C(F)(F)F)C(F)(F)F)cc3Cl)c2)cnc1Cl)N(CC1CC1)C1CC1] | 100 | 100 | 76.7 |
| 4 | 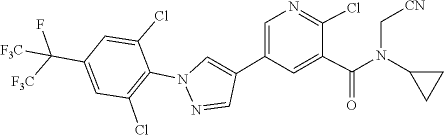 [SMILES: N#CCN(C(=O)c1cc(-c2cnn(-c3c(Cl)cc(C(F)(C(F)(F)F)C(F)(F)F)cc3Cl)c2)cnc1Cl)C1CC1] | 100 | 100 | 100 |
| 5 | 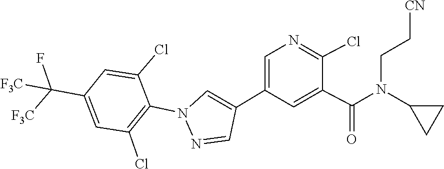 [SMILES: N#CCCN(C(=O)c1cc(-c2cnn(-c3c(Cl)cc(C(F)(C(F)(F)F)C(F)(F)F)cc3Cl)c2)cnc1Cl)C1CC1] | 100 | 100 | 100 |
| 99 | 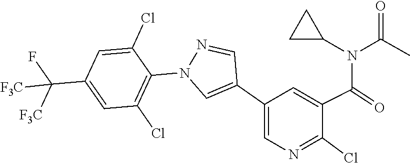 [SMILES: CC(=O)N(C(=O)c1cc(-c2cnn(-c3c(Cl)cc(C(F)(C(F)(F)F)C(F)(F)F)cc3Cl)c2)cnc1Cl)C1CC1] | 100 | 100 | 100 |
| 104 | 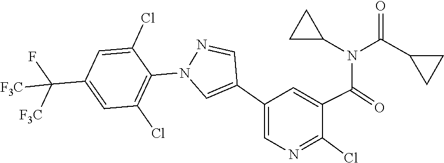 [SMILES: O=C(c1cc(-c2cnn(-c3c(Cl)cc(C(F)(C(F)(F)F)C(F)(F)F)cc3Cl)c2)cnc1Cl)N(C(=O)C1CC1)C1CC1] | 100 | 100 | 100 |
| 105 | 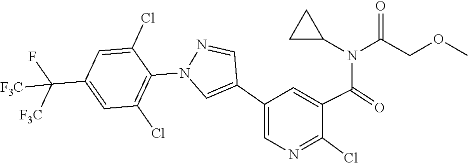 [SMILES: COCC(=O)N(C(=O)c1cc(-c2cnn(-c3c(Cl)cc(C(F)(C(F)(F)F)C(F)(F)F)cc3Cl)c2)cnc1Cl)C1CC1] | 100 | 100 | 100 |
| CK1 | 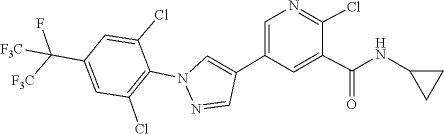 [SMILES: O=C(NC1CC1)c1cc(-c2cnn(-c3c(Cl)cc(C(F)(C(F)(F)F)C(F)(F)F)cc3Cl)c2)cnc1Cl] | 100 | 66.7 | 20 |
| CK2 | 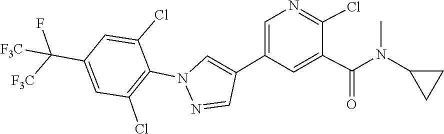 [SMILES: CN(C(=O)c1cc(-c2cnn(-c3c(Cl)cc(C(F)(C(F)(F)F)C(F)(F)F)cc3Cl)c2)cnc1Cl)C1CC1] | 93.3 | 60 | 13.3 |
| 11 | 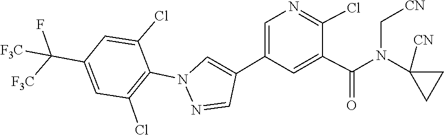 [SMILES: N#CCN(C(=O)c1cc(-c2cnn(-c3c(Cl)cc(C(F)(C(F)(F)F)C(F)(F)F)cc3Cl)c2)cnc1Cl)C1(C#N)CC1] | 100 | 100 | 100 |
| 12 | 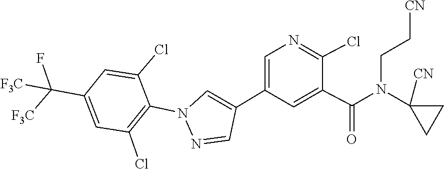 [SMILES: N#CCCN(C(=O)c1cc(-c2cnn(-c3c(Cl)cc(C(F)(C(F)(F)F)C(F)(F)F)cc3Cl)c2)cnc1Cl)C1(C#N)CC1] | 100 | 100 | 100 |
| 108 | 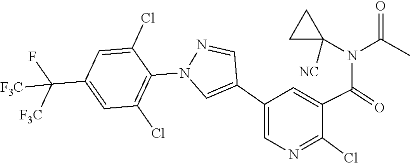 [SMILES: CC(=O)N(C(=O)c1cc(-c2cnn(-c3c(Cl)cc(C(F)(C(F)(F)F)C(F)(F)F)cc3Cl)c2)cnc1Cl)C1(C#N)CC1] | 100 | 100 | 100 |
| 113 | 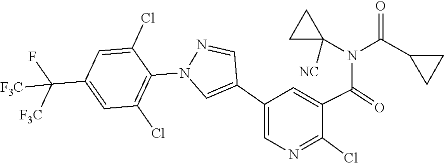 [SMILES: N#CC1(N(C(=O)c2cc(-c3cnn(-c4c(Cl)cc(C(F)(C(F)(F)F)C(F)(F)F)cc4Cl)c3)cnc2Cl)C(=O)C2CC2)CC1] | 100 | 100 | 100 |
| 114 | 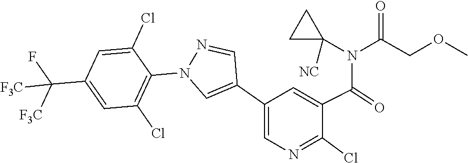 [SMILES: COCC(=O)N(C(=O)c1cc(-c2cnn(-c3c(Cl)cc(C(F)(C(F)(F)F)C(F)(F)F)cc3Cl)c2)cnc1Cl)C1(C#N)CC1] | 100 | 100 | 100 |
| CK3 | 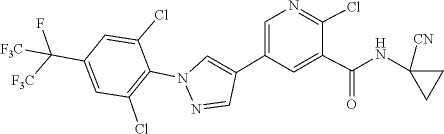 [SMILES: N#CC1(NC(=O)c2cc(-c3cnn(-c4c(Cl)cc(C(F)(C(F)(F)F)C(F)(F)F)cc4Cl)c3)cnc2Cl)CC1] | 100 | 60 | 30 |
| CK4 | 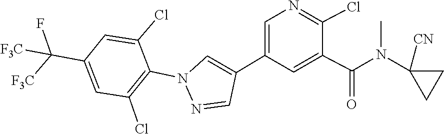 [SMILES: CN(C(=O)c1cc(-c2cnn(-c3c(Cl)cc(C(F)(C(F)(F)F)C(F)(F)F)cc3Cl)c2)cnc1Cl)C1(C#N)CC1] | 90 | 56.7 | 20 |

Note: CK1, CK2, CK3, CK4 in the table are all the control compounds provided in the present disclosure, these control compounds can be obtained by referring to the method in Embodiment 1 or Embodiment 7 of the present disclosure, and all of raw materials can be prepared by methods in the embodiments of the present disclosure or can be purchased or prepared by conventional methods.

In the embodiments of the present disclosure, compounds with better insecticidal effects were obtained by selecting W₁, W₂, Y₁, Y₂, R₁, Rₐ and combinations thereof in the compound as shown in the formula I. As shown in Table 5, by comparing compounds 1, 2, 3, 4, 5, 99, 104 and 105 with control compounds CK1 and CK2 and by comparing compounds 11, 12, 108, 113 and 114 with control compounds CK3 and CK4, it can be seen that since R₁ in the compound as shown in the general formula I of the present disclosure is selected from allyl, propargyl, cyclopropylmethyl, CNCH₂—, CNCH₂CH₂—, CNCH₂CH₂CH₂—, CNCH₂CH₂CH₂CH₂—, or

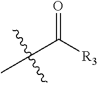

and R₃ is selected from C₁-C₄ alkyl, C₃-C₆ cycloalkyl, or C₁-C₄ alkoxy C₁-C₃ alkyl, the compound of the present disclosure exhibits unexpected high insecticidal activity.

### Embodiment 12: Biological Activity Determination of Tetranychus cinnabarinus

The greenhouse acaricidal activity determination was carried out on of compounds of the present disclosure. The determination method is as follows:

According to the solubility of the compounds to be tested, the compounds to be tested were dissolved in acetone or dimethyl sulfoxide and prepared into 50 mL of solutions to be tested at desired concentrations with a 0.1% Tween 80 solution, where the content of the acetone or dimethyl sulfoxide in the solutions was not higher than 10%.

Two true leaves of Phaseolus vulgaris seedlings were taken, and 30-40 adult acarids of Tetranychus cinnabarinus were inoculated. After the cardinal number was investigated, the whole plants were subjected to spraying treatment with a hand-held sprayer, each treatment was repeated for 3 times, and the plants were placed in a standard observation room after the treatment. The number of surviving acarids was investigated after 72 h, and the mortality was calculated.

Some test results of adult acarids of Tetranychus cinnabarinus are as follows:

At the dose of 1.25 mg/L, the mortality of compounds 1, 2, 3, 4, 5, 11, 12, 18, 32, 33, 34, 46, 60, 74, 88, 99, 101, 102, 104, 105, 108, 109, 110, 111, 113 and 114 against adult acarids of Tetranychus cinnabarinus is 90% or above.

### Embodiment 13: Insecticidal Test on Ctenocephalides felis

4 mg of compounds to be tested were dissolved in 40 mL of acetone to obtain acetone solutions with a concentration of 100 mg/L. Bottom surfaces and side surfaces of culture dishes with an inner diameter of 5.3 cm were coated with 400 μL of the drug solutions, and when the acetone was volatilized, films of the compounds of the present disclosure were prepared on inner walls of the culture dishes. The inner walls of the culture dishes used were 40 cm², and a dose of the treatment drugs was 1 μg/cm². 10 adult Ctenocephalides felis (males and females were mixed) were placed in the culture dishes, and the culture dishes were covered and then stored in a constant-temperature room at 25° C. The number of dead insects was checked after 72 h, and the insect mortality was calculated. The test was repeated for 3 times. Test results: The insect mortality of compounds 1, 2, 3, 4, 5, 11, 12, 18, 32, 33, 34, 46, 60, 74, 88, 99, 101, 102, 104, 105, 108, 109, 110, 111, 113 and 114 is 70% or above.

### Embodiment 14: Insecticidal Test on Dermacentor variabilis

4 mg of compounds to be tested were dissolved in 40 mL of acetone to obtain acetone solutions with a concentration of 100 mg/L. Bottom surfaces and side surfaces of 2 culture dishes with an inner diameter of 5.3 cm were coated with 400 μL of the drug solutions, and when the acetone was volatilized, films of the compounds of the present disclosure were prepared on inner walls of the culture dishes. The inner walls of the culture dishes used were 40 cm², and a dose of the treatment drugs was 1 μg/cm². 10 1st nymphs of Dermacentor variabilis (males and females were mixed) were placed in the culture dishes. The 2 culture dishes were combined, sealed with tape at a junction portion to prevent escape of the nymphs, and then stored in a constant-temperature room at 25° C. The number of dead insects was checked after 24 h, and the insect mortality was calculated. The test was repeated for 3 times. Test results: The insect mortality of compounds 1, 2, 3, 4, 5, 11, 12, 18, 32, 33, 34, 46, 60, 74, 88, 99, 101, 102, 104, 105, 108, 109, 110, 111, 113 and 114 is 70% or above.

1. A phenylpyrazole compound, wherein a structure of the phenylpyrazole compound is as shown in general formula I, or is a stereoisomer of the compound as shown in the general formula I, a salt of the compound as shown in the general formula I, or a salt of the stereoisomer of the compound as shown in the general formula I: 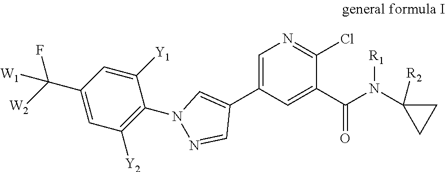
wherein, in the general formula I:
W₁ is selected from fluorine, trifluoromethyl, or pentafluoroethyl;
W₂ is selected from fluorine or trifluoromethyl;
Y₁ is selected from halogen;
Y₂ is selected from halogen, C₁-C₃ haloalkyl, or C₁-C₃ haloalkoxy;
R₁ is selected from allyl, propargyl, cyclopropylmethyl, CNCH₂—, CNCH₂CH₂—, CNCH₂CH₂CH₂—, CNCH₂CH₂CH₂CH₂—, or 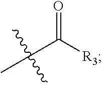
R₂ is selected from hydrogen or cyanide; and
R₃ is selected from C₁-C₄ alkyl, C₃-C₆ cycloalkyl, or C₁-C₄ alkoxy C₁-C₃ alkyl.

2. The phenylpyrazole compound according to claim 1, wherein in the general formula I:
W₁ is selected from fluorine, trifluoromethyl, or pentafluoroethyl;
W₂ is selected from fluorine or trifluoromethyl;
Y₁ is selected from chlorine or bromine;
Y₂ is selected from chlorine, bromine, C₁-C₃ haloalkyl, or C₁-C₃ haloalkoxy;
R₁ is selected from allyl, propargyl, cyclopropylmethyl, CNCH₂—, CNCH₂CH₂—, CNCH₂CH₂CH₂—, CNCH₂CH₂CH₂CH₂—, or 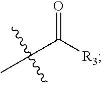
R₂ is selected from hydrogen or cyanide; and
R₃ is selected from methyl, ethyl, n-propyl, isopropyl, n-butyl, isobutyl, sec-butyl, tert-butyl, cyclopropyl, cyclobutyl, cyclopentyl, cyclohexyl, CH₃OCH₂—, CH₃CH₂OCH₂—, CH₃CH₂CH₂OCH₂—, (CH₃)₂CHOCH₂—, CH₃CH₂CH₂CH₂OCH₂—, (CH₃)₃COCH₂—, CH₃OCH₂CH₂—CH₃CH₂OCH₂CH₂—, CH₃CH₂CH₂OCH₂CH₂—, or CH₃CH₂CH₂CH₂OCH₂CH₂—.

3. The phenylpyrazole compound according to claim 2, wherein in the general formula I:
W₁ is selected from fluorine, trifluoromethyl, or pentafluoroethyl;
W₂ is selected from fluorine or trifluoromethyl;
Y₁ is selected from chlorine or bromine;
Y₂ is selected from chlorine, bromine, trifluoromethyl, difluoromethoxy, or trifluoromethoxy;
R₁ is selected from allyl, propargyl, cyclopropylmethyl, CNCH₂—, CNCH₂CH₂—, CNCH₂CH₂CH₂—, CNCH₂CH₂CH₂CH₂—, or 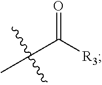
R₂ is selected from hydrogen or cyanide; and
R₃ is selected from methyl, ethyl, n-propyl, isopropyl, cyclopropyl, CH₃OCH₂—, CH₃CH₂OCH₂—, or CH₃CH₂CH₂OCH₂—.

4. The phenylpyrazole compound according to claim 1, wherein in the general formula I:
when W₁ and W₂ are selected from trifluoromethyl and R₁ is selected from allyl, propargyl, cyclopropylmethyl, CNCH₂—, CNCH₂CH₂—, CNCH₂CH₂CH₂—, or CNCH₂CH₂CH₂CH₂—, the phenylpyrazole compound is selected from compounds in Table 1, wherein the compounds in the Table 1 have the structure as shown in the general formula I, and Y₁, Y₂, R₁, and R₂ are as shown in the Table 1:
TABLE 1

| Compound | | | | |
|---|---|---|---|---|
| Number | Y₁ | Y₂ | R₁ | R₂ |
| 1 | Cl | Cl | Allyl | H |
| 2 | C | Cl | Propargyl | H |
| 3 | Cl | Cl | Cyclopropylmethyl | H |
| 4 | Cl | Cl | CNCH₂— | H |
| 5 | Cl | Cl | CNCH₂CH₂— | H |
| 6 | Cl | Cl | CNCH₂CH₂CH₂— | H |
| 7 | Cl | Cl | CNCH₂CH₂CH₂CH₂— | H |
| 8 | Cl | Cl | Allyl | CN |
| 9 | Cl | Cl | Propargyl | CN |
| 10 | Cl | Cl | Cyclopropylmethyl | CN |
| 11 | Cl | Cl | CNCH₂— | CN |
| 12 | Cl | Cl | CNCH₂CH₂— | CN |
| 13 | Cl | Cl | CNCH₂CH₂CH₂— | CN |
| 14 | Cl | Cl | CNCH₂CH₂CH₂CH₂— | CN |
| 15 | Br | Br | Allyl | H |
| 16 | Br | Br | Propargyl | H |
| 17 | Br | Br | Cyclopropylmethyl | H |
| 18 | Br | Br | CNCH₂— | H |
| 19 | Br | Br | CNCH₂CH₂— | H |
| 20 | Br | Br | CNCH₂CH₂CH₂— | H |
| 21 | Br | Br | CNCH₂CH₂CH₂CH₂— | H |
| 22 | Br | Br | Allyl | CN |
| 23 | Br | Br | Propargyl | CN |
| 24 | Br | Br | Cyclopropylmethyl | CN |
| 25 | Br | Br | CNCH₂— | CN |
| 26 | Br | Br | CNCH₂CH₂— | CN |
| 27 | Br | Br | CNCH₂CH₂CH₂— | CN |
| 28 | Br | Br | CNCH₂CH₂CH₂CH₂— | CN |
| 29 | Br | Cl | Allyl | H |
| 30 | Br | Cl | Propargyl | H |
| 31 | Br | Cl | Cyclopropylmethyl | H |
| 32 | Br | Cl | CNCH₂— | H |
| 33 | Br | Cl | CNCH₂CH₂— | H |
| 34 | Br | Cl | CNCH₂CH₂CH₂— | H |
| 35 | Br | Cl | CNCH₂CH₂CH₂CH₂— | H |
| 36 | Br | Cl | Ally₁ | CN |
| 37 | Br | Cl | Propargyl | CN |
| 38 | Br | Cl | Cyclopropylmethyl | CN |
| 39 | Br | Cl | CNCH₂— | CN |
| 40 | Br | Cl | CNCH₂CH₂— | CN |
| 41 | Br | Cl | CNCH₂CH₂CH₂— | CN |
| 42 | Br | Cl | CNCH₂CH₂CH₂CH₂— | CN |
| 43 | Br | CF₃ | Allyl | H |
| 44 | Br | CF₃ | Propargyl | H |
| 45 | Br | CF₃ | Cyclopropylmethyl | H |
| 46 | Br | CF₃ | CNCH₂— | H |
| 47 | Br | CF₃ | CNCH₂CH₂— | H |
| 48 | Br | CF₃ | CNCH₂CH₂CH₂— | H |
| 49 | Br | CF₃ | CNCH₂CH₂CH₂CH₂— | H |
| 50 | Br | CF₃ | Allyl | CN |
| 51 | Br | CF₃ | Propargyl | CN |
| 52 | Br | CF₃ | Cyclopropylmethyl | CN |
| 53 | Br | CF₃ | CNCH₂— | CN |
| 54 | Br | CF₃ | CNCH₂CH₂— | CN |
| 55 | Br | CF₃ | CNCH₂CH₂CH₂— | CN |
| 56 | Br | CF₃ | CNCH₂CH₂CH₂CH₂— | CN |
| 57 | Cl | CF₃ | Allyl | H |
| 58 | Cl | CF₃ | Propargyl | H |
| 59 | Cl | CF₃ | Cyclopropylmethyl | H |
| 60 | Cl | CF₃ | CNCH₂— | H |
| 61 | Cl | CF₃ | CNCH₂CH₂— | H |
| 62 | Cl | CF₃ | CNCH₂CH₂CH₂— | H |
| 63 | Cl | CF₃ | CNCH₂CH₂CH₂CH₂— | H |
| 64 | Cl | CF₃ | Allyl | CN |
| 65 | Cl | CF₃ | Propargyl | CN |
| 66 | Cl | CF₃ | Cyclopropylmethyl | CN |
| 67 | Cl | CF₃ | CNCH₂— | CN |
| 68 | Cl | CF₃ | CNCH₂CH₂— | CN |
| 69 | Cl | CF₃ | CNCH₂CH₂CH₂— | CN |
| 70 | Cl | CF₃ | CNCH₂CH₂CH₂CH₂— | CN |
| 71 | Br | OCF₃ | Allyl | H |
| 72 | Br | OCF₃ | Propargyl | H |
| 73 | Br | OCF₃ | Cyclopropylmethyl | H |
| 74 | Br | OCF₃ | CNCH₂— | H |
| 75 | Br | OCF₃ | CNCH₂CH₂— | H |
| 76 | Br | OCF₃ | CNCH₂CH₂CH₂— | H |
| 77 | Br | OCF₃ | CNCH₂CH₂CH₂CH₂— | H |
| 78 | Br | OCF₃ | Allyl | CN |
| 79 | Br | OCF₃ | Propargyl | CN |
| 80 | Br | OCF₃ | Cyclopropylmethyl | CN |
| 81 | Br | OCF₃ | CNCH₂— | CN |
| 82 | Br | OCF₃ | CNCH₂CH₂— | CN |
| 83 | Br | OCF₃ | CNCH₂CH₂CH₂— | CN |
| 84 | Br | OCF₃ | CNCH₂CH₂CH₂CH₂— | CN |
| 85 | Cl | OCF₃ | Allyl | H |
| 86 | Cl | OCF₃ | Propargyl | H |
| 87 | Cl | OCF₃ | Cyclopropylmethyl | H |
| 88 | Cl | OCF₃ | CNCH₂— | H |
| 89 | Cl | OCF₃ | CNCH₂CH₂— | H |
| 90 | Cl | OCF₃ | CNCH₂CH₂CH₂— | H |
| 91 | Cl | OCF₃ | CNCH₂CH₂CH₂CH₂— | H |
| 92 | Cl | OCF₃ | Allyl | CN |
| 93 | Cl | OCF₃ | Propargyl | CN |
| 94 | Cl | OCF₃ | Cyclopropylmethyl | CN |
| 95 | Cl | OCF₃ | CNCH₂— | CN |
| 96 | Cl | OCF₃ | CNCH₂CH₂— | CN |
| 97 | Cl | OCF₃ | CNCH₂CH₂CH₂— | CN |
| 98 | Cl | OCF₃ | CNCH₂CH₂CH₂CH₂— | CN |

5. The phenylpyrazole compound according to claim 1, wherein in the general formula I:
when W₁ and W₂ are selected from trifluoromethyl and R₁ is selected from 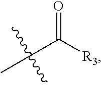
the phenylpyrazole compound is selected from compounds in Table 2, wherein the compounds in the Table 2 have the structure as shown in the general formula I, and Y₁, Y₂, R₂, and R₃ are as shown in the Table 2:
TABLE 2

| Compound | | | | |
|---|---|---|---|---|
| Number | Y₁ | Y₂ | R₂ | R₃ |
| 99 | Cl | Cl | H | Methyl |
| 100 | Cl | Cl | H | Ethyl |
| 101 | Cl | Cl | H | N-propyl |
| 102 | Cl | Cl | H | Isopropyl |
| 103 | Cl | Cl | H | N-butyl |
| 104 | Cl | Cl | H | Cyclopropyl |
| 105 | Cl | Cl | H | CH₃OCH₂— |
| 106 | Cl | Cl | H | CH₃CH₂OCH₂— |
| 107 | Cl | Cl | H | CH₃CH₂CH₂OCH₂— |
| 108 | Cl | Cl | CN | Methyl |
| 109 | Cl | Cl | CN | Ethyl |
| 110 | Cl | Cl | CN | N-propyl |
| 111 | Cl | Cl | CN | Isopropyl |
| 112 | Cl | Cl | CN | N-butyl |
| 113 | Cl | Cl | CN | Cyclopropyl |
| 114 | Cl | Cl | CN | CH₃OCH₂— |
| 115 | Cl | Cl | CN | CH₃CH₂OCH₂— |
| 116 | Cl | Cl | CN | CH₃CH₂CH₂OCH₂— |
| 117 | Br | Br | H | Methyl |
| 118 | Br | Br | H | Ethyl |
| 119 | Br | Br | H | N-propyl |
| 120 | Br | Br | H | Isopropyl |
| 121 | Br | Br | H | N-butyl |
| 122 | Br | Br | H | Cyclopropyl |
| 123 | Br | Br | H | CH₃OCH₂— |
| 124 | Br | Br | H | CH₃CH₂OCH₂— |
| 125 | Br | Br | H | CH₃CH₂CH₂OCH₂— |
| 126 | Br | Br | CN | Methyl |
| 127 | Br | Br | CN | Ethyl |
| 128 | Br | Br | CN | N-propyl |
| 129 | Br | Br | CN | Isopropyl |
| 130 | Br | Br | CN | N-butyl |
| 131 | Br | Br | CN | Cyclopropyl |
| 132 | Br | Br | CN | CH₃OCH₂— |
| 133 | Br | Br | CN | CH₃CH₂OCH₂— |
| 134 | Br | Br | CN | CH₃CH₂CH₂OCH₂— |
| 135 | Br | Cl | H | Methyl |
| 136 | Br | Cl | H | Ethyl |
| 137 | Br | Cl | H | N-propyl |
| 138 | Br | Cl | H | Isopropyl |
| 139 | Br | Cl | H | N-butyl |
| 140 | Br | Cl | H | Cyclopropyl |
| 141 | Br | Cl | H | CH₃OCH₂— |
| 142 | Br | Cl | H | CH₃CH₂OCH₂— |
| 143 | Br | Cl | H | CH₃CH₂CH₂OCH₂— |
| 144 | Br | Cl | CN | Methyl |
| 145 | Br | Cl | CN | Ethyl |
| 146 | Br | Cl | CN | N-propyl |
| 147 | Br | Cl | CN | Isopropyl |
| 148 | Br | Cl | CN | N-butyl |
| 149 | Br | Cl | CN | Cyclopropyl |
| 150 | Br | Cl | CN | CH₃OCH₂— |
| 151 | Br | Cl | CN | CH₃CH₂OCH₂— |
| 152 | Br | Cl | CN | CH₃CH₂CH₂OCH₂— |
| 153 | Br | CF₃ | H | Methyl |
| 154 | Br | CF₃ | H | Ethyl |
| 155 | Br | CF₃ | H | N-propyl |
| 156 | Br | CF₃ | H | Isopropyl |
| 157 | Br | CF₃ | H | N-butyl |
| 158 | Br | CF₃ | H | Cyclopropyl |
| 159 | Br | CF₃ | H | CH₃OCH₂— |
| 160 | Br | CF₃ | H | CH₃CH₂OCH₂— |
| 161 | Br | CF₃ | H | CH₃CH₂CH₂OCH₂— |
| 162 | Br | CF₃ | CN | Methyl |
| 163 | Br | CF₃ | CN | Ethyl |
| 164 | Br | CF₃ | CN | N-propyl |
| 165 | Br | CF₃ | CN | Isopropyl |
| 166 | Br | CF₃ | CN | N-butyl |
| 167 | Br | CF₃ | CN | Cyclopropyl |
| 168 | Br | CF₃ | CN | CH₃OCH₂— |
| 169 | Br | CF₃ | CN | CH₃CH₂OCH₂— |
| 170 | Br | CF₃ | CN | CH₃CH₂CH₂OCH₂— |
| 171 | Cl | CF₃ | H | Methyl |
| 172 | Cl | CF₃ | H | Ethyl |
| 173 | Cl | CF₃ | H | N-propyl |
| 174 | Cl | CF₃ | H | Isopropyl |
| 175 | Cl | CF₃ | H | N-butyl |
| 176 | Cl | CF₃ | H | Cyclopropyl |
| 177 | Cl | CF₃ | H | CH₃OCH₂— |
| 178 | Cl | CF₃ | H | CH₃CH₂OCH₂— |
| 179 | Cl | CF₃ | H | CH₃CH₂CH₂OCH₂— |
| 180 | Cl | CF₃ | CN | Methyl |
| 181 | Cl | CF₃ | CN | Ethyl |
| 182 | Cl | CF₃ | CN | N-propyl |
| 183 | Cl | CF₃ | CN | Isopropyl |
| 184 | Cl | CF₃ | CN | N-butyl |
| 185 | Cl | CF₃ | CN | Cyclopropyl |
| 186 | Cl | CF₃ | CN | CH₃OCH₂— |
| 187 | Cl | CF₃ | CN | CH₃CH₂OCH₂— |
| 188 | Cl | CF₃ | CN | CH₃CH₂CH₂OCH₂— |
| 189 | Br | OCF₃ | H | Methyl |
| 190 | Br | OCF₃ | H | Ethyl |
| 191 | Br | OCF₃ | H | N-propyl |
| 192 | Br | OCF₃ | H | Isopropyl |
| 193 | Br | OCF₃ | H | N-butyl |
| 194 | Br | OCF₃ | H | Cyclopropyl |
| 195 | Br | OCF₃ | H | CH₃OCH₂— |
| 196 | Br | OCF₃ | H | CH₃CH₂OCH₂— |
| 197 | Br | OCF₃ | H | CH₃CH₂CH₂OCH₂— |
| 198 | Br | OCF₃ | CN | Methyl |
| 199 | Br | OCF₃ | CN | Ethyl |
| 200 | Br | OCF₃ | CN | N-propyl |
| 201 | Br | OCF₃ | CN | Isopropyl |
| 202 | Br | OCF₃ | CN | N-butyl |
| 203 | Br | OCF₃ | CN | Cyclopropyl |
| 204 | Br | OCF₃ | CN | CH₃OCH₂— |
| 205 | Br | OCF₃ | CN | CH₃CH₂OCH₂— |
| 206 | Br | OCF₃ | CN | CH₃CH₂CH₂OCH₂— |
| 207 | Cl | OCF₃ | H | Methyl |
| 208 | Cl | OCF₃ | H | Ethyl |
| 209 | Cl | OCF₃ | H | N-propyl |
| 210 | Cl | OCF₃ | H | Isopropyl |
| 211 | Cl | OCF₃ | H | N-butyl |
| 212 | Cl | OCF₃ | H | Cyclopropyl |
| 213 | Cl | OCF₃ | H | CH₃OCH₂— |
| 214 | Cl | OCF₃ | H | CH₃CH₂OCH₂— |
| 215 | Cl | OCF₃ | H | CH₃CH₂CH₂OCH₂— |
| 216 | Cl | OCF₃ | CN | Methyl |
| 217 | Cl | OCF₃ | CN | Ethyl |
| 218 | Cl | OCF₃ | CN | N-propyl |
| 219 | Cl | OCF₃ | CN | Isopropyl |
| 220 | Cl | OCF₃ | CN | N-butyl |
| 221 | Cl | OCF₃ | CN | Cyclopropyl |
| 222 | Cl | OCF₃ | CN | CH₃OCH₂— |
| 223 | Cl | OCF₃ | CN | CH₃CH₂OCH₂— |
| 224 | Cl | OCF₃ | CN | CH₃CH₂CH₂OCH₂— |

6. The phenylpyrazole compound according to claim 1, wherein in the general formula I:
when W₁ and W₂ are selected from trifluoromethyl and R₁ is selected from allyl, propargyl, cyclopropylmethyl, CNCH₂—, CNCH₂CH₂—, CNCH₂CH₂CH₂—, or CNCH₂CH₂CH₂CH₂—, the phenylpyrazole compound is selected from compounds in Table 3, wherein the compounds in the Table 3 have the structure as shown in the general formula I, and Y₁, Y₂, R₁, and R₂ are as shown in the Table 3:
TABLE 3

| Compound | | | | |
|---|---|---|---|---|
| Number | Y₁ | Y₂ | R₁ | R₂ |
| 1 | Cl | Cl | Allyl | H |
| 2 | Cl | Cl | Propargyl | H |
| 3 | Cl | Cl | Cyclopropylmethyl | H |
| 4 | Cl | Cl | CNCH₂— | H |
| 5 | Cl | Cl | CNCH₂CH₂— | H |
| 11 | Cl | Cl | CNCH₂— | CN |
| 12 | Cl | Cl | CNCH₂CH₂— | CN |
| 18 | Br | Br | CNCH₂— | H |
| 19 | Br | Br | CNCH₂CH₂— | H |
| 25 | Br | Br | CNCH₂— | CN |
| 26 | Br | Br | CNCH₂CH₂— | CN |
| 32 | Br | Cl | CNCH₂— | H |
| 33 | Br | Cl | CNCH₂CH₂— | H |
| 34 | Br | Cl | CNCH₂CH₂CH₂— | H |
| 39 | Br | Cl | CNCH₂— | CN |
| 40 | Br | Cl | CNCH₂CH₂— | CN |
| 46 | Br | CF₃ | CNCH₂— | H |
| 47 | Br | CF₃ | CNCH₂CH₂— | H |
| 53 | Br | CF₃ | CNCH₂— | CN |
| 54 | Br | CF₃ | CNCH₂CH₂— | CN |
| 60 | Cl | CF₃ | CNCH₂— | H |
| 61 | Cl | CF₃ | CNCH₂CH₂— | H |
| 67 | Cl | CF₃ | CNCH₂— | CN |
| 68 | Cl | CF₃ | CNCH₂CH₂— | CN |
| 74 | Br | OCF₃ | CNCH₂— | H |
| 75 | Br | OCF₃ | CNCH₂CH₂— | H |
| 81 | Br | OCF₃ | CNCH₂— | CN |
| 82 | Br | OCF₃ | CNCH₂CH₂— | CN |
| 88 | Cl | OCF₃ | CNCH₂— | H |
| 89 | Cl | OCF₃ | CNCH₂CH₂— | H |
| 95 | Cl | OCF₃ | CNCH₂— | CN |
| 96 | Cl | OCF₃ | CNCH₂CH₂— | CN |

7. The phenylpyrazole compound according to claim 1, wherein in the general formula I:
when W₁ and W₂ are selected from trifluoromethyl and R₁ is selected from 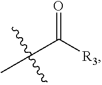
the phenylpyrazole compound is selected from compounds in Table 4, wherein the compounds in the Table 4 have the structure as shown in the general formula I, and Y₁, Y₂, R₂, and R₃ are as shown in the Table 4:
TABLE 4

| Compound | | | | |
|---|---|---|---|---|
| Number | Y₁ | Y₂ | R₂ | R₃ |
| 99 | Cl | Cl | H | Methyl |
| 100 | Cl | Cl | H | Ethyl |
| 101 | Cl | Cl | H | N-propyl |
| 103 | Cl | Cl | H | N-butyl |
| 104 | Cl | Cl | H | Cyclopropyl |
| 105 | Cl | Cl | H | CH₃OCH₂— |
| 106 | Cl | Cl | H | CH₃CH₂OCH₂— |
| 107 | Cl | Cl | H | CH₃CH₂CH₂OCH₂— |
| 108 | Cl | Cl | CN | Methyl |
| 109 | Cl | Cl | CN | Ethyl |
| 110 | Cl | Cl | CN | N-propyl |
| 112 | Cl | Cl | CN | N-butyl |
| 113 | Cl | Cl | CN | Cyclopropyl |
| 114 | Cl | Cl | CN | CH₃OCH₂— |
| 115 | Cl | Cl | CN | CH₃CH₂OCH₂— |
| 116 | Cl | Cl | CN | CH₃CH₂CH₂OCH₂—. |

8. The phenylpyrazole compound according to claim 1, wherein the salt of the compound as shown in the general formula I comprises: a salt formed by the compound as shown in the general formula I and hydrochloric acid, hydrobromic acid, sulfuric acid, phosphoric acid, formic acid, acetic acid, propaonic acid, butyric acid, valeric acid, trifluoroacetic acid, oxalic acid, propanedioic acid, methanesulfonic acid, 4-toluenesulfonic acid, malic acid, fumaric acid, lactic acid, maleic acid, salicylic acid, tartaric acid, or citric acid;
and/or, the salt of the stereoisomer of the compound as shown in the general formula I comprises: a salt formed by the stereoisomer of the compound as shown in the general formula I and hydrochloric acid, hydrobromic acid, sulfuric acid, phosphoric acid, formic acid, acetic acid, propaonic acid, butyric acid, valeric acid, trifluoroacetic acid, oxalic acid, propanedioic acid, methanesulfonic acid, 4-toluenesulfonic acid, malic acid, fumaric acid, lactic acid, maleic acid, salicylic acid, tartaric acid, or citric acid.

9. A use of the phenylpyrazole compound according to claim 1 in preparation of an insecticide and/or an acaricide.

10. The use according to claim 9, wherein the insecticide and/or the acaricide is used for preventing and controlling one or more of Mythimna separata, Plutella xylostella, and Tetranychus cinnabarinus.

11. An insecticide or acaricide formulation, wherein the insecticide or acaricide formulation comprises the phenylpyrazole compound according to claim 1 as an active component, and further comprises one or more excipients; and optionally, an amount of the phenylpyrazole compound according to claim 1 in the insecticide or acaricide formulation is 0.1 to 99 wt %, further optionally 0.5 to 90 wt %.

12. An insecticide or acaricide composition, comprising a mixture of the phenylpyrazole compound according to claim 1 and other active compounds, wherein the other active compounds are selected from one or more of an insecticide, a poison bait, a disinfectant, an acaricide, a nematicide, a fungicide, a growth regulator, and a herbicide.

13. A method for controlling agricultural or forestry pests and/or acarids, comprising administering an effective dose of a material to the pests, the acarids, or a growth medium thereof to be controlled, wherein the material is selected from one or more of the following groups:
the phenylpyrazole compound according to claim 1;
an insecticide or acaricide formulation, comprising the phenylpyrazole compound according to claim 1 as an active component, and further comprises one or more excipients, and optionally, an amount of the phenylpyrazole compound according to claim 1 in the insecticide or acaricide formulation is 0.1 to 99 wt %, further optionally 0.5 to 90 wt %; and
an insecticide or acaricide composition, comprising a mixture of the phenylpyrazole compound according to claim 1 and other active compounds, wherein the other active compounds are selected from one or more of an insecticide, a poison bait, a disinfectant, an acaricide, a nematicide, a fungicide, a growth regulator, and a herbicide.

14. A use of the phenylpyrazole compound according to claim 1 in preparation of an animal parasite control agent.

15. The use according to claim 14, wherein the animal parasite control agent is used for preventing and controlling one or more of Ctenocephalides felis and Dermacentor variabilis.

16. An animal parasite control agent, wherein the animal parasite control agent comprises the phenylpyrazole compound according to claim 1 as an active component, and further comprises one or more excipients; and optionally, an amount of the phenylpyrazole compound according to claim 1 in the animal parasite control agent is 1 to 80 wt %.

17. An animal parasite control composition, comprising a mixture of the phenylpyrazole compound according to claim 1 and other animal parasite control active compounds, wherein the other animal parasite control active compounds are selected from one or more of an acaricide, an insecticide, a parasiticide, and an antimalarial agent.

18. A method for controlling animal parasites, comprising the following step: administering an effective dose of a material to the animal parasites or a growth medium thereof to be controlled, wherein the material is selected from one or more of the following groups:
the phenylpyrazole compound according to claim 1;
an animal parasite control agent, comprising the phenylpyrazole compound according to claim 1 as an active component, and further comprises one or more excipients, and optionally, an amount of the phenylpyrazole compound according to claim 1 in the animal parasite control agent is 1 to 80 wt %; and
an animal parasite control composition, comprising a mixture of the phenylpyrazole compound according to claim 1 and other animal parasite control active compounds, wherein the other animal parasite control active compounds are selected from one or more of an acaricide, an insecticide, a parasiticide, and an antimalarial agent.
